(19) United States
(12) Patent Application Publication
(10) Pub. No.: US 2021/0254069 A1
(43) Pub. Date: Aug. 19, 2021

(54) COMBINATION THERAPIES COMPRISING C/EBP ALPHA SARNA

(71) Applicant: MiNA THERAPEUTICS LIMITED, London (GB)

(72) Inventors: Helen L Lightfoot, London (GB); Vikash Reebye, London (GB); Pål Sætrom, Trondheim (NO); David Blakey, London (GB); Choon Ping Tan, London (GB)

(21) Appl. No.: 17/252,593

(22) PCT Filed: Jun. 14, 2019
(86) PCT No.: PCT/GB2019/051654

§ 371 Date: Dec. 15, 2020

Related U.S. Application Data
(60) Provisional application No. 62/685,627, filed on Jun. 15, 2018.
(60) Provisional application No. 62/731,532, filed on Sep. 14, 2018.
(60) Provisional application No. 62/821,533, filed on Mar. 21, 2019.

(51) Int. Cl. C12N 15/113; C07K 14/47; A61K 31/7088; C07K 16/28; A61K 31/44; A61K 31/47; A61P 35/00

(52) U.S. Cl. CPC C12N 15/113; C07K 14/4705; A61K 31/7088; C12N 15/1138; C07K 16/2863; C07K 16/2818; A61K 2039/505; A61K 31/47; A61P 35/00; C12N 2310/113; C12N 2320/31; C12N 2310/14; C07K 2317/76; A61K 31/44

(57) ABSTRACT

The invention relates to a combination therapy comprising an saRNA targeting C/EBPα and at least one additional active agent. Methods of using the combination therapy are also provided.

### CROSS-REFERENCE TO RELATED APPLICATIONS

This application claims priority to U.S. Prov. Application Ser. No. 62/685,627 filed Jun. 15, 2018, entitled, “COMBINATION THERAPIES COMPRISING C/EBP ALPHA SARNA”, U.S. Prov. Application Ser. No. 62/731,532 filed Sep. 14, 2018, entitled, “COMBINATION THERAPIES COMPRISING C/EBP ALPHA SARNA”, and U.S Prov. Application Ser. No. 62/821,533 filed Mar. 21, 2019, entitled “COMBINATION THERAPIES COMPRISING C/EBP ALPHA SARNA”, the contents of each of which are incorporated herein by reference in their entirety.

### REFERENCE TO SEQUENCE LISTING

The present application is being filed along with a Sequence Listing in electronic format. The sequence listing filed in ASCII format, entitled, 2058-1024PCT_SEQ_LIST.txt, was created on May 30, 2019 and is 8,905 bytes in size. The information in electronic format of the Sequence Listing is incorporated herein by reference in its entirety.

### FIELD OF THE DISCLOSURE

The invention relates to polynucleotide, specifically saRNA, compositions for the modulating C/EBPα and C/EBPα pathways and to the methods of using the compositions in therapeutic applications such as treating metabolic disorders, hyperproliferative diseases including cancer, and regulating stem cell linage.

### BACKGROUND OF THE DISCLOSURE

CCAAT/enhancer-binding protein a (C/EBPα, C/EBP alpha, C/EBPA or CEBPA) is a leucine zipper protein that is conserved across humans and rats. This nuclear transcription factor is enriched in hepatocytes, myelomonocytes, adipocytes, as well as other types of mammary epithelial cells [Lekstrom-Himes et al., J. Bio. Chem, vol. 273, 28545-28548 (1998)]. It is composed of two transactivation domains in the N-terminal part, and a leucine zipper region mediating dimerization with other C/EBP family members and a DNA-binding domain in the C-terminal part. The binding sites for the family of C/EBP transcription factors are present in the promoter regions of numerous genes that are involved in the maintenance of normal hepatocyte function and response to injury. C/EBPα has a pleiotropic effect on the transcription of several liver-specific genes implicated in the immune and inflammatory responses, development, cell proliferation, anti-apoptosis, and several metabolic pathways [Darlington et al., Current Opinion of Genetic Development, vol. 5(5), 565-570 (1995)]. It is essential for maintaining the differentiated state of hepatocytes. It activates albumin transcription and coordinates the expression of genes encoding multiple ornithine cycle enzymes involved in urea production, therefore playing an important role in normal liver function.

In the adult liver, C/EBPα is defined as functioning in terminally differentiated hepatocytes whilst rapidly proliferating hepatoma cells express only a fraction of C/EBPa [Umek et al., Science, vol. 251, 288-292 (1991)]. C/EBPα is known to up-regulate p21, a strong inhibitor of cell proliferation through the up-regulation of retinoblastoma and inhibition of Cdk2 and Cdk4 [Timchenko et al., Genes & Development, vol. 10, 804-815 (1996); Wang et al., Molecular Cell, vol. 8, 817-828 (2001)]. In hepatocellular carcinoma (HCC), C/EBPα functions as a tumor suppressor with anti-proliferative properties [Iakova et al., Seminars in Cancer Biology, vol. 21(1), 28-34 (2011)].

Different approaches are carried out to study C/EBPα mRNA or protein modulation. It is known that C/EBPα protein is regulated by post-translational phosphorylation and sumoylation. For example, FLT3 tyrosine kinase inhibitors and extra-cellular signal-regulated kinases 1 and/or 2 (ERK1/2) block serine-21 phosphorylation of C/EBPα, which increases the granulocytic differentiation potential of the C/EBPα protein [Radomska et al., Journal of Experimental Medicine, vol. 203(2), 371-381 (2006) and Ross et al., Molecular and Cellular Biology, vol. 24(2), 675-686 (2004)]. In addition, C/EBPα translation can be efficiently induced by 2-cyano-3,12-dioxoolean-1,9-dien-28-oic acid (CDDO), which alters the ratio of the C/EBPα protein isoforms in favor of the full-length p42 form over p30 form thereby inducing granulocytic differentiation [Koschmieder et al., Blood, vol. 110(10), 3695-3705 (2007)].

The C/EBPα gene is an intronless gene located on chromosome 19q13.1. Most eukaryotic cells use RNA-complementarity as a mechanism for regulating gene expression. One example is the RNA interference (RNAi) pathway which uses double stranded short interfering RNAs to knockdown gene expression via the RNA-induced silencing complex (RISC). It is now established that short duplex RNA oligonucleotides also have the ability to target the promoter regions of genes and mediate transcriptional activation of these genes and they have been referred to as RNA activation (RNAa), antigene RNA (agRNA) or short activating RNA (saRNA) [Li et al., PNAS, vol. 103, 17337-17342 (2006)]. saRNA induced activation of genes is conserved in other mammalian species including mouse, rat, and non-human primates and is fast becoming a popular method for studying the effects of endogenous up-regulation of genes.

Thus, there is a need for targeted modulation of C/EBPα for therapeutic purposes with saRNA.

### SUMMARY OF THE DISCLOSURE

The present disclosure provides combinational therapies comprising CEBPA-saRNA molecules and at least one additional active agent. Methods of preparing and using the combinational therapies are also provided.

The details of various embodiments of the invention are set forth in the description below. Other features, objects, and advantages of the invention will be apparent from the description and the drawings, and from the claims.

### BRIEF DESCRIPTION OF THE DRAWINGS

FIG. 1 is a schematic illustrating the relationships among the nucleic acid moieties involved in the function of an saRNA of the invention.

FIG. 2 is a timeline of study design of Example 4.

FIG. 3 shows changes in tumour infiltrating helper T lymphocytes as discussed in Example 4.

FIG. 4 shows changes in tumour infiltrating cytotoxic T lymphocytes as discussed in Example 4.

FIG. 5 shows changes in tumour infiltrating Natural Killer T cells without RFA treatment as discussed in Example 4.

FIG. 6 shows changes in tumour infiltrating Natural Killer T cells with RFA treatment as discussed in Example 4.

FIG. 7A shows tumor volume changes as discussed in Example 5.

FIG. 7B shows AFP changes as discussed in Example 5.

FIG. 8A, 8B, 8C and 8D shows CT26 tumour size for each animal in group over duration of the study as well as a scatter plots at day 18, 21 and 23 in a study discussed in Example 7.

FIG. 9A shows tumour weights at Week 3 of MTL-CEBA+ Nexavar (left panel) and MTL-CEBPA+anti-PD1 (right panel) treated animals

FIG. 9B shows tumour volumes at Week 3 of MTL-CEBA+anti-PD1 (top panel) and MTL-CEBPA+Nexavar (bottom panel) treated animals.

### DETAILED DESCRIPTION

The present invention provides compositions, methods and kits for modulating C/EBPα gene expression and/or function for therapeutic purposes. These compositions, methods and kits comprise nucleic acid constructs that target a C/EBPα transcript.

C/EBPα protein is known as a critical regulator of metabolic processes and cell proliferation. Modulating C/EBPα gene has great potentials for therapeutic purposes. The present invention addresses this need by providing nucleic acid constructs targeting a C/EBPα transcript, wherein the nucleic acid constructs may include single or double stranded DNA or RNA with or without modifications.

C/EBPα gene as used herein is a double-stranded DNA comprising a coding strand and a template strand. It may also be referred to the target gene in the present application.

The terms “C/EBPα transcript”, “C/EBPα target transcript” or “target transcript” in the context may be C/EBPα mRNA encoding C/EBPα protein. C/EBPα mRNA is transcribed from the template strand of C/EBPα gene and may exist in the mitochondria.

The antisense RNA of the C/EBPα gene transcribed from the coding strand of the C/EBPα gene is called a target antisense RNA transcript herein after. The target antisense RNA transcript may be a long non-coding antisense RNA transcript.

The terms “small activating RNA”, “short activating RNA”, or “saRNA” in the context of the present invention means a single-stranded or double-stranded RNA that upregulates or has a positive effect on the expression of a specific gene. The saRNA may be single-stranded of 14 to 30 nucleotides. The saRNA may also be double-stranded, each strand comprising 14 to 30 nucleotides. The gene is called the target gene of the saRNA. A saRNA that upregulates the expression of the C/EBPα gene is called a “C/EBPα-saRNA” and the C/EBPα gene is the target gene of the C/EBPα-saRNA.

The terms “target” or “targeting” in the context mean having an effect on a C/EBPα gene. The effect may be direct or indirect. Direct effect may be caused by complete or partial hybridization with the C/EBPα target antisense RNA transcript. Indirect effect may be upstream or downstream.

C/EBPα-saRNA may have a downstream effect on a biological process or activity. In such embodiments, C/EBPα-saRNA may have an effect (either upregulating or downregulating) on a second, non-target transcript.

The term “gene expression” in the context may include the transcription step of generating C/EBPα mRNA from C/EBPα gene or the translation step generating C/EBPα protein from C/EBPα mRNA. An increase of C/EBPα mRNA and an increase of C/EBPα protein both indicate an increase or a positive effect of C/EBPα gene expression.

By “upregulation” or “activation” of a gene is meant an increase in the level of expression of a gene, or levels of the polypeptide(s) encoded by a gene or the activity thereof, or levels of the RNA transcript(s) transcribed from the template strand of a gene above that observed in the absence of the saRNA of the present invention. The saRNA of the present invention may have a direct or indirect upregulating effect on the expression of the target gene.

In one embodiment, the saRNA of the present invention may show efficacy in proliferating cells. As used herein with respect to cells, “proliferating” means cells which are growing and/or reproducing rapidly.

### I. Composition of the Invention

One aspect of the present invention provides pharmaceutical compositions comprising a saRNA that upregulates CEBPA gene, and at least one pharmaceutically acceptable carrier. Such a saRNA is referred herein after as “C/EBPα-saRNA”, or “saRNA of the present invention”, used interchangeably in this application.

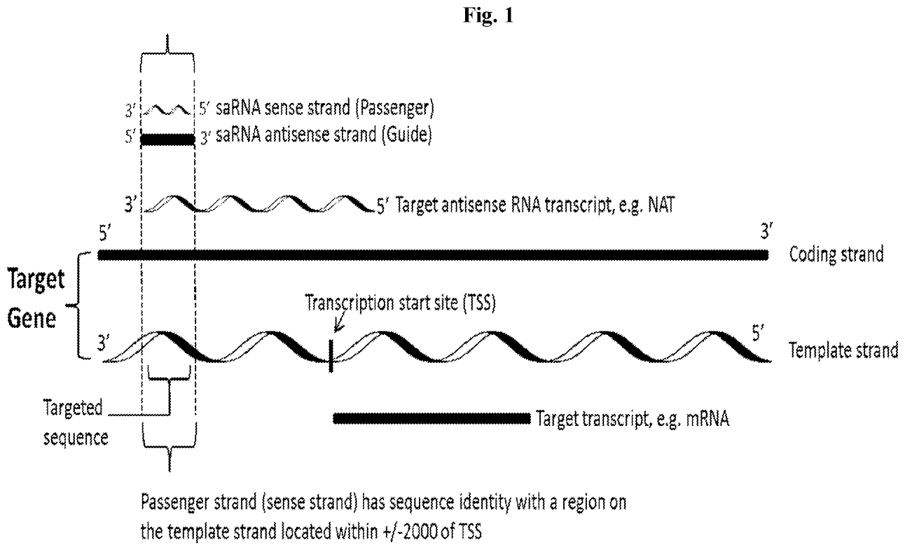

The C/EBPα-saRNA has 14-30 nucleotides and comprises a sequence that is at least 80%, 90%, 95%, 98%, 99% or 100% complementary to a targeted sequence on the template strand of the C/EBPα gene. The targeted sequence may have the same length, i.e., the same number of nucleotides, as the saRNA and/or the reverse complement of the saRNA. The relationships among the saRNAs, a target gene, a coding strand of the target gene, a template strand of the target gene, a targeted sequence/target site, and the transcription start site (TSS) are shown in FIG. 1.

In some embodiments, the targeted sequence comprises at least 14 and less than 30 nucleotides.

In some embodiments, the targeted sequence has 19, 20, 21, 22, or 23 nucleotides.

In some embodiments, the location of the targeted sequence is situated within a promoter area of the template strand.

In some embodiments, the targeted sequence of the C/EBPα-saRNA is located within a TSS (transcription start site) core of the template stand of the C/EBPα gene. A “TSS core” or “TSS core sequence” as used herein, refers to a region between 2000 nucleotides upstream and 2000 nucleotides downstream of the TSS (transcription start site). Therefore, the TSS core comprises 4001 nucleotides and the TSS is located at position 2001 from the 5′ end of the TSS core sequence. CEBPA TSS core sequence is show in the table below:

| | | CEBPA TSS core | CEBPA TSS core |
|---|---|---|---|
| CEBPA mRNA REF. No. | CEBPA protein REF. No. | genomic location | sequence ID No. |
| NM_001285829 | NP_001272758 | chr19:33302564 | SEQ ID No. 3 |
| NM_001287424 | NP_001274353 | minus strand | |
| NM_001287435 | NP_001274364 | | |
| NM_004364 | NP_004355 | | |

In some embodiments, the targeted sequence is located between 1000 nucleotides upstream and 1000 nucleotides downstream of the TSS.

In some embodiments, the targeted sequence is located between 500 nucleotides upstream and 500 nucleotides downstream of the TSS.

In some embodiments, the targeted sequence is located between 250 nucleotides upstream and 250 nucleotides downstream of the TSS.

In some embodiments, the targeted sequence is located between 100 nucleotides upstream and 100 nucleotides downstream of the TSS.

In some embodiments, the targeted sequence is located upstream of the TSS in the TSS core. The targeted sequence may be less than 2000, less than 1000, less than 500, less than 250, or less than 100 nucleotides upstream of the TSS.

In some embodiments, the targeted sequence is located downstream of the TSS in the TSS core. The targeted sequence may be less than 2000, less than 1000, less than 500, less than 250, or less than 100 nucleotides downstream of the TSS.

In some embodiments, the targeted sequence is located +/- 50 nucleotides surrounding the TSS of the TSS core. In some embodiments, the targeted sequence substantially overlaps the TSS of the TSS core. In some embodiments, the targeted sequence begins or ends at the TSS of the TSS core. In some embodiments, the targeted sequence overlaps the TSS of the TSS core by 1, 2, 3, 4, 5, 6, 7, 8, 9, 10, 11, 12, 13, 14, 15, 16, 17, 18 or 19 nucleotides in either the upstream or downstream direction.

The location of the targeted sequence on the template strand is defined by the location of the 5′ end of the targeted sequence. The 5′ end of the targeted sequence may be at any position of the TSS core and the targeted sequence may start at any position selected from position 1 to position 4001 of the TSS core. For reference herein, when the 5′ most end of the targeted sequence from position 1 to position 2000 of the TSS core, the targeted sequence is considered upstream of the TSS and when the 5′ most end of the targeted sequence is from position 2002 to 4001, the targeted sequence is considered downstream of the TSS. When the 5′ most end of the targeted sequence is at nucleotide 2001, the targeted sequence is considered to be a TSS centric sequence and is neither upstream nor downstream of the TSS.

For further reference, for example, when the 5′ end of the targeted sequence is at position 1600 of the TSS core, i.e., it is the 1600^{th} nucleotide of the TSS core, the targeted sequence starts at position 1600 of the TSS core and is considered to be upstream of the TSS.

In one embodiment, the saRNA of the present invention may have two strands that form a duplex, one strand being a guide strand. The saRNA duplex is also called a double-stranded saRNA. A double-stranded saRNA or saRNA duplex, as used herein, is a saRNA that includes more than one, and preferably, two, strands in which interstrand hybridization can form a region of duplex structure. The two strands of a double-stranded saRNA are referred to as an antisense strand or a guide strand, and a sense strand or a passenger strand.

In some embodiments, the C/EBPα-saRNA may comprising any C/EBPα-saRNA disclosed in WO2015/075557 or WO2016/170349 to MiNA Therapeutics Limited, the contents of each of which are incorporated herein by reference in their entirety, such as saRNAs in Table 1, Table 1A, Table 3-1 and Table 3-2, AW51, and CEBPA-51 disclosed in WO2016/170349.

In some embodiments, the C/EBPα-saRNA may be modified and may comprising any modification disclosed in WO2016/170349 to MiNA Therapeutics Limited.

In one embodiment, the C/EBPα-saRNA is CEBPA-51 (or CEBPA51), which is an saRNA duplex that upregulates C/EBPa. Its design, sequences, and compositions/formulations are disclosed in the Detailed Description and Examples of WO2016/170349 to MiNA Therapeutics Limited. The sequences of the sense and antisense strands of CEBPA-51 are shown in Table 1.

TABLE 1
CEBPA-51 (CEBPA51) Sequences

| | | |
|---|---|---|
| Antisense | GACCAGUGACAAUGACCGCmUmU | SEQ ID No. 1 |
| Sense | (invabasic)mGmCGmGUCAUUmGUCAmCUGGUCmUmU | SEQ ID No. 2 |

mU, mG, and mC mean 2′-O-methyl modified U, G, and C.
invabasic = inverted abasic sugar cap.

The alignment of the strands is shown in the Table 2.

TABLE 2
CEBPA-51 Alignment of Strands

| saRNA name | CEBPA-51 Total base: 21 mer including base modifications | | | | | | |
|---|---|---|---|---|---|---|---|
| mer | 3 | 6 | 9 | 12 | 15 | 18 | 21 |
| Sense strand | bmGmCG | mGUC | AUU | mGUC | AmCU | GGU | CmUmU |
| 5′ → 3′ | | | | | | | |
| (SEQ ID No. 2) | | | | | | | |
| Complementary | mUmUC | GCC | AGU | AAC | AGU | GAC | CAG |
| antisense strand | | | | | | | |
| 3′ → 5′ | | | | | | | |
| (SEQ ID No. 1) | | | | | | | |

Definition of symbols: A, U, G, C are 2′-OH ribonucleotides, mU, mG, mC are 2′-O-methyl ribonucleotides,
b = inverted abasic sugar cap.

CEBPA-51 is encapsulated into liposomes (NOV340 SMARTICLES technology owned by Marina Biotech) to make MTL-CEBPA. The lipid components of the NOV340 SMARTICLES® are comprised of 1-palmitoyl-2-oleoyl-sn-glycero-3-phosphocholine (POPC), 1,2-dioleoyl-sn-glycero-3-phosphoethanolamine (DOPE), cholesteryl-hemi succinate (CHEMS), and 4-(2-aminoethyl)-morpholino-cholesterol hemi succinate (MOCHOL). NOV340 SMARTICLES® consists of POPC, DOPE, CHEMS and MOCHOL in the molar ratio of 6:24:23:47. These nanoparticles are anionic at physiological pH, and their specific lipid ratio imparts a “pH-tunable” character and a charge to the liposomes, which changes depending upon the surrounding pH of the microenvironment to facilitate movement across physiologic membranes. SMARTICLES® nanoparticles are sized to avoid extensive immediate hepatic sequestration, with an average diameter of approximately about 50—about 150 nm, or about 100—about 120 nm, facilitating more prolonged systemic distribution and improved serum stability after i.v. injection leading to broader tissue distribution with high levels in liver, spleen and bone marrow reported.

MTL-CEBPA also comprises the buffer forming excipients such as sucrose and phosphate-salts. Qualitative and quantitative composition of MTL-CEBPA (2.5 mg/ml) are shown in Table 3.

TABLE 3
MTL-CEBPA Composition

| | | | Quantity |
|---|---|---|---|
| Name of Ingredient | Function | Reference | (per ml) |
| CEBPA-51 (saRNA) | Active pharmaceutical | Manufacturer's | 2.5 mg/ml |
| | ingredient | specifications | |
| 1-palmitoyl-2-oleoyl-sn-glycero-3- | Membrane forming lipid | Manufacturer's | 4.65 mg/ml |
| phosphocholine (POPC) | | specifications | |
| 1,2-dioleoyl-sn-glycero-3- | Membrane forming | Manufacturer's | 18.0 mg/ml |
| phosphoethanolamine (DOPE) | fusogenic lipid | specifications | |
| Cholesteryl hemisuccinate (CHEMS) | Anionic ampotheric lipid | Manufacturer's | 11.3 mg/ml |
| | | specifications | |
| Cholesteryl-4-[[2-(4- | Cationic amphoteric lipid | Manufacturer's | 27.0 mg/ml |
| morpholinyl)ethyl]amino]-4-oxobutanoate | | specifications | |
| (MOCHOL) | | | |
| Sucrose | Cryoprotectant, | BP, JP, NF, EP | 92.4 mg/ml |
| | osmolality control | | |
| Disodium hydrogen phosphate, dihydrate | Buffer pH adjustment | BP, USP, EP | 1.44 mg/ml |
| Potassium dihydrogen phosphate | Buffer pH adjustment | EP, BP, NF | 0.2 mg/ml |
| Potassium chloride (KCl) | Ionic strength adjuster | EP, BP, USP | 0.2 mg/ml |
| Water for injection (WFI) | Solvent | WFI (USP, EP) | qs 1 ml |

Administration

C/EBPα-saRNAs or C/EBPα-saRNA compositions, such as CEBPA-51 and/or MTL-CEBPA, may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them to cross the blood-brain barrier, vascular barrier, or other epithelial barrier. Routes of administration disclosed in International Publication WO 2013/090648 filed Dec. 14, 2012, the contents of which are incorporated herein by reference in their entirety, may be used to administer the saRNA of the present invention.

Dosing

In some embodiments, C/EBPα-saRNAs or C/EBPα-saRNA compositions, such as CEBPA-51 and/or MTL-CEBPA, are administered once every day, once every 2 days, once every 3 days, once every 4 days, or once every 5 days.

In some embodiments, at least two doses of C/EBPα-saRNAs or C/EBPα-saRNA compositions, such as CEBPA-51 and/or MTL-CEBPA, are administered to a subject. The subject may have a liver disease, such as liver cancer, non-alcoholic steatohepatitis (NASH), steatosis, liver damage, liver failure, or liver fibrosis. The doses are less than 7 days apart. In one embodiment, CEBPA-51 and/or MTL-CEBPA is administered every 24 hours. In one embodiment, CEBPA-51 and/or MTL-CEBPA is administered every 48 hours.

In some embodiments, the patient receives at least 2 doses, e.g, 3 doses, 4 doses, 5 doses, 6 doses, 7 doses, 8 doses, 9 doses, or 10 doses, of C/EBPα-saRNAs or C/EBPα-saRNA compositions, such as CEBPA-51 and/or MTL-CEBPA.

In some embodiments, C/EBPα-saRNAs or C/EBPα-saRNA compositions, such as CEBPA-51 and/or MTL-CEBPA, are administered for a period of at least 2 days, such as 3 days, 4 days, 5 days, 6 days, 1 week, 8 days, 9 days, 10 days, 2 weeks, 3 weeks, 4 weeks, 5 weeks, or 6 weeks.

In one embodiment, CEBPA-51 and/or MTL-CEBPA is administered every 24 hours for a period of at least 2 days, such as 3 days, 4 days, 5 days, 6 days, 1 week, 8 days, 9 days, 10 days, 2 weeks, 3 weeks, 4 weeks, 5 weeks, or 6 weeks.

In one embodiment, CEBPA-51 and/or MTL-CEBPA is administered every 48 hours for a period of at least 2 days, such as 3 days, 4 days, 5 days, 6 days, 1 week, 8 days, 9 days, 10 days, 2 weeks, 3 weeks, 4 weeks, 5 weeks, or 6 weeks.

In some embodiments, C/EBPα-saRNAs or C/EBPα-saRNA compositions, such as CEBPA-51 and/or MTL-CEBPA, are administered via intravenous infusion over 60 minutes. Doses are between about 20 to about 160 mg/m².

The dosing regimen disclosed in the present application may apply to any indication or disorder that can be treated with C/EBPα-saRNAs or C/EBPα-saRNA compositions.

### II. Methods of Use

One aspect of the present invention provides methods of using C/EBPα-saRNA and pharmaceutical compositions comprising said C/EBPα-saRNA and at least one pharmaceutically acceptable carrier. C/EBPα-saRNA modulates C/EBPα gene expression. In one embodiment, the expression of C/EBPα gene is increased by at least 20, 30, 40%, more preferably at least 45, 50, 55, 60, 65, 70, 75%, even more preferably at least 80% in the presence of the saRNA of the present invention compared to the expression of C/EBPα gene in the absence of the saRNA of the present invention. In a further preferable embodiment, the expression of C/EBPα gene is increased by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of the saRNA of the present invention compared to the expression of C/EBPα gene in the absence of the saRNA of the present invention.

In one embodiment, the increase in gene expression of the saRNA descried herein is shown in proliferating cells.

### Metabolics Regulation

Hepatocytes are generally perceived as being important for maintenance of several vital functions. For example, they can regulate carbohydrate and lipid metabolism and detoxification of exogenous and endogenous compounds. C/EBPα is expressed in a variety of tissues where it plays an important role in the differentiation of many cell types including adipocytes, type II alveolar cells and hepatocytes. In the mouse, C/EBPα is found most abundantly in fat, liver and lung tissues. The function role of C/EBPα includes, but not limited to, regulation of alpha-1-antitrypsin, transthyretin and albumin. Furthermore, expression of C/EBPα gene in the liver cell line (HepG2) results in increased levels of cytochrome P450 (CYP), a superfamily of monooxygenases that participates in the metabolism of endogenous substrates and plays a key role in detoxification and metabolic activation of key xenobiotics [Jover et al., FEBS Letters, vol. 431(2), 227-230 (1998), the contents of which are incorporated herein by reference in their entirety].

Non-alcoholic fatty liver disease (NAFLD) is a major global health concern and affects 1 in 3 people in the United States. NAFLD is the build-up of extra fat (lipid) in liver cells that is not caused by excessive alcohol use. It is called a fatty liver (steatosis) if more than 5%-10% of the liver's weight is fat. NAFLD may progress to steatoheptitis, cirrhosis, and liver cancer. It is associated with metabolic disorders, such as metabolic syndrome, insulin resistance, type II diabetes, hyperlipidemia, hypertension, obesity, etc. Treatment methods include lowering low-density lipoprotein (LDL) cholesterol levels, improving insulin sensitivity, treating metabolic risk factors, weight loss and so on. [Adams et al., Postgraduate Medical Journal, vol. 82, 315-322 (2006); Musso et al., Curr. Opin. Lipidol., vol. 22(6), 489-496 (2011), the contents of which are incorporated herein by reference in their entirety]

C/EBPα protein plays an important role in regulating liver function and metabolics. The primary effects of C/EBPα on the liver are shown in FIG. 1, including decreasing fatty acid uptake by lowering CD36 protein level, decreasing de novo lipogenesis by lowering sterol regulatory element-binding proteins (SREBP), carbohydrate-responsive element-binding protein (ChREBP) and fatty acid synthase (FAS) protein levels, increasing β-oxidation by increasing peroxisome proliferator-activated receptor alpha (PPARα) and peroxisome proliferator-activated receptor gamma coactivator 1-alpha & -beta (PGC-1α & β) protein levels, decreasing hepatic lipid overload by lowering apolipoprotein C-III (APOC3) and low density lipoprotein receptor (LDLR) protein levels, decreasing progression to fibrosis by increasing PGC-1β protein level, and decreasing insulin resistance by increasing peroxisome proliferator-activated receptor gamma (PPARγ) protein level. Furthermore, C/EBPα has secondary effects on adipose tissues. White adipose tissue (WAT) is not only a lipogenic and fat storage tissue but also an important endocrine organ that regulates energy homeostasis, lipid metabolism, appetite, fertility, and immune and stress responses. Brown adipose tissue (BAT) contains numerous smaller lipid droplets and a much higher number of iron-containing mitochondria compared with WAT. It plays a significant role in nutritional energetics, energy balance and body weight. There is evidence that the atrophy of BAT is related to obesity. In particular, studies have indicated that impaired thermogenesis in BAT is important in the aetiology of obesity in rodents [Trayhurn P., J. Biosci., vol. 18(2), 161-173 (1993)]. C/EBPα decreases hepatic steatosis and insulin resistance and increases PGC-1α protein level, which may in turn cause browning of WAT, turn WAT into BAT, and then activate BAT, thereby reducing body fat and weight. Therefore, C/EBPα-saRNA of the present invention may be used to regulate liver function, reduce steatosis, reduce serum lipids, treat NAFLD, treat insulin resistance, increase energy expenditure, and treat obesity.

In one embodiment, provided is a method of regulating liver metabolism genes in vitro and in vivo by treatment of C/EBPα-saRNA of the present invention. Also provided is a method of regulating liver genes involved in NAFLD in vitro and in vivo by treatment of C/EBPα-saRNA of the present invention. The genes include, but are not limited to sterol regulatory element-binding factor 1 (SREBF-1 or SREBF), cluster of differentiation 36 (CD36), acetyl-CoA carboxylase 2 (ACACB), apolipoprotein C-III (APOC3), microsomal triglyceride transfer protein (MTP), peroxisome proliferator-activated receptor gamma coactivator 1 alpha (PPARγ-CoA1α or PPARGC1A), low density lipoprotein receptor (LDLR), peroxisome proliferator-activated receptor gamma coactivator 1 beta (PPARγ-CoA1β or PERC), peroxisome proliferator-activated receptor gamma (PPARγ), acetyl-CoA carboxylase 1 (ACACA), carbohydrate-responsive element-binding protein (ChREBP or MLX1PL), peroxisome proliferator-activated receptor alpha (PPARα or PPARA), FASN (fatty acid synthase), diglyceride acyltransferase-2 (DGAT2), and mammalian target of rapamycin (mTOR). In one embodiment, C/EBPα-saRNA decreases the expression of SREBF-1 gene in liver cells by at least 20%, 30%, preferably at least 40%. In one embodiment, C/EBPα-saRNA decreases the expression of CD36 gene in liver cells by at least 20%, 30%, 40%, 50%, preferably at least 75%, 90%. In one embodiment, C/EBPα-saRNA increases the expression of ACACB gene in liver cells by at least 20%, 30%, 40%, 50%, preferably at least 75%, 90%, 100%, 125%, 150%. In one embodiment, C/EBPα-saRNA decreases the expression of APOC3 gene in liver cells by at least 20%, 30%, 40%, 50%, preferably at least 75%, 90%. In one embodiment, C/EBPα-saRNA decreases the expression of MTP gene in liver cells by at least 20%, 30%, 40%, 50%, preferably at least 75%, 90%. In one embodiment, C/EBPα-saRNA increases the expression of PPARγ-CoA1α gene in liver cells by at least 20%, 30%, 40%, 50%, preferably at least 75%, 90%, 100%, 125%, 150%, more preferably at least 175%, 200%, 250%, 300%. In one embodiment, C/EBPα-saRNA increases the expression of PPARγ gene in liver cells by at least 20%, 30%, 40%, 50%, preferably at least 75%, 90%, 100%, 125%, 150%, more preferably at least 175%, 200%, 250%, 300%. In one embodiment, C/EBPα-saRNA increases the expression of PPARα gene in liver cells by at least 20%, 30%, 40%, 50%, preferably at least 75%, 90%, 100%, 125%, 150%, more preferably at least 175%, 200%, 250%, 300%. In one embodiment, C/EBPα-saRNA decreases the expression of MLXIPL gene in liver cells by at least 20%, 30%, 40%, 50%, preferably at least 75%. In one embodiment, C/EBPα-saRNA decreases the expression of FASN gene in liver cells by at least 20%, 30%, 40%, 50%, preferably at least 75%, 90%. In one embodiment, C/EBPα-saRNA decreases the expression of DGAT2 gene in liver cells by at least 10%, 20%, preferably at least 30%, 40%, 50%.

C/EBPα-saRNA also modulates the expression of liver metabolism genes disclosed above in BAT cells. In another embodiment, C/EBPα-saRNA decreases the expression of SREBP gene in BAT cells by at least 20%, 30%, preferably at least 40%. In one embodiment, C/EBPα-saRNA decreases the expression of CD36 gene in BAT cells by at least 20%, 30%, 40%, 50%, preferably at least 75%, 90%. In one embodiment, C/EBPα-saRNA decreases the expression of LDLR gene in BAT cells by at least 20%, 30%, 40%, 50%, preferably at least 75%, 90%. In one embodiment, C/EBPα-saRNA increases the expression of PPARGC1A gene in BAT cells by at least 20%, 30%, preferably at least 40%. In one embodiment, C/EBPα-saRNA decreases the expression of APOC gene in BAT cells by at least 20%, 30%, 40%, 50%, preferably at least 75%, 90%, more preferably at least 95%, 99%. In one embodiment, C/EBPα-saRNA decreases the expression of ACACB gene in BAT cells by at least 20%, 30%, 40%, 50%, preferably at least 75%. In one embodiment, C/EBPα-saRNA decreases the expression of PERC gene in BAT cells by at least 20%, 30%, 40%, 50%, preferably at least 75%. In one embodiment, C/EBPα-saRNA increases the expression of ACACA gene in BAT cells by at least 20%, 30%, 40%, 50%, preferably at least 75%, 90%, 100%, 125%, 150%. In one embodiment, C/EBPα-saRNA decreases the expression of MLXP1 gene in BAT cells by at least 20%, 30%, 40%, preferably at least 50%. In one embodiment, C/EBPα-saRNA decreases the expression of MTOR gene in BAT cells by at least 20%, 30%, 40%, preferably at least 50%, 75%. In one embodiment, C/EBPα-saRNA increases the expression of PPARA gene in BAT cells by at least 20%, 30%, 40%, 50%, preferably at least 75%, 90%, 100%, 125%, 150%, more preferably at least 200%, 250%, 300%, 350%, 400%. In one embodiment, C/EBPα-saRNA increases the expression of FASN gene in BAT cells by at least 20%, 30%, 40%, 50%, preferably at least 75%, 90%. In one embodiment, C/EBPα-saRNA increases the expression of DGAT gene in BAT cells by at least 20%, 30%, 40%, 50%, preferably at least 75%, 90%, 100%, 125%, 150%, more preferably at least 200%, 250%, 300%.

C/EBPα-saRNA also modulates the expression of liver metabolism genes disclosed above in WAT cells. In another embodiment, C/EBPα-saRNA decreases the expression of SREBP gene in WAT cells by at least 20%, 30%, preferably at least 40%. In one embodiment, C/EBPα-saRNA decreases the expression of CD36 gene in WAT cells by at least 20%, 30%, 40%, 50%, preferably at least 75%, 90%. In one embodiment, C/EBPα-saRNA decreases the expression of LDLR gene in WAT cells by at least 20%, 30%, 40%, 50%, preferably at least 75%, 90%. In one embodiment, C/EBPα-saRNA increases the expression of PPARGC1A gene in WAT cells by at least 20%, 30%, preferably at least 40%. In one embodiment, C/EBPα-saRNA increases the expression of MTP gene in WAT cells by at least 20%, 30%, 40%, 50%, preferably at least 75%, 90%, more preferably at least 95%, more preferably at least by a factor of 1.5, 2.0, 2.5, 3.0, 3.5, 4.0, more preferably by at least a factor of 5.0, 6.0, 7.0, 8.0, 9.0, 10.0. In one embodiment, In one embodiment, C/EBPα-saRNA increases the expression of APOC gene in WAT cells by at least 20%, 30%, 40%, 50%, preferably at least 75%, 90%, more preferably at least 95%, 99%. In one embodiment, C/EBPα-saRNA decreases the expression of ACACB gene in WAT cells by at least 20%, 30%, 40%, 50%, preferably at least 75%. In one embodiment, C/EBPα-saRNA decreases the expression of PERC gene in WAT cells by at least 20%, 30%, 40%, 50%, preferably at least 75%. In one embodiment, C/EBPα-saRNA decreases the expression of ACACA gene in WAT cells by at least 20%, 30%, 40%, 50%, preferably at least 75%, 90%, 95%. In one embodiment, C/EBPα-saRNA decreases the expression of MLX1PL gene in WAT cells by at least 20%, 30%, 40%, preferably at least 50%. In one embodiment, C/EBPα-saRNA decreases the expression of MTOR gene in WAT cells by at least 20%, 30%, 40%, preferably at least 50%, 75%. In one embodiment, C/EBPα-saRNA decreases the expression of FASN gene in WAT cells by at least 5%, 10%, preferably at least 15%, 20%. In one embodiment, C/EBPα-saRNA decreases the expression of DGAT gene in WAT cells by at least 10%, 20%, 30%, more preferably 40%, 50%.

In another embodiment, provided is a method of reducing insulin resistance (IR) or increasing insulin sensitivity by administering C/EBPα-saRNA of the present invention to a patient in need thereof. Also provided is a method of treating type II diabetes, hyperinsulinaemia and steatosis by administering C/EBPα-saRNA of the present invention to a patient in need thereof If liver cells are resistance to insulin and cannot use insulin effectively, hyperglycemia develops. Subsequently, beta cells in pancreas increase their production of insulin leading to hyperinsulinemia and type II diabetes. Many regulators affect insulin resistance of liver cells. For example, sterol regulatory element-binding proteins 1 (SREBP1 or SREBP) is the master regulator of cholesterol and associated with increased insulin resistance. The up-regulation of cholesteryl ester transfer protein (CETP) is associated with increased insulin resistance. The up-regulation of hepatic fatty acid translocase/cluster of differentiation 36 (FAT/CD36) is associated with insulin resistance, hyperinsulinaemia, increased steatosis in patients with non-alcoholic steatohepatitis (NASH). Liver-specific overexpression of lipoprotein lipase gene (LPL) causes liver-specific insulin resistance. Liver X receptor gene (LXR) has a central role in insulin-mediated activation of sterol regulatory element-binding protein (SREBP)-1c-induced fatty acid synthesis in liver. Other factors include diglyceride acyltransferase-2 (DGAT2) that regulates triglyceride synthesis and fatty acid synthase (FASN) that regulates fatty acid biosynthesis. In one embodiment, C/EBPα-saRNA reduces the expression of FAT/CD36 gene in liver cells by at least 25%, preferably at least 50%, more preferably at least 75%, even more preferably 90% compared to liver cells with no treatment. In another embodiment, C/EBPα-saRNA increases the expression of LPL gene in liver cells by at least 20, 30, 40%, preferably at least 45, 50, 55, 60, 65, 70, 75, 80, 85, 90, 95%, more preferably at least 100, 150, 200, 250, 300, 350 and 400% compared to liver cells with no treatment. In another embodiment, C/EBPα-saRNA increases the expression of LXR gene in liver cells by at least 45, 50, 55, 60, 65, 70, 75, 80, 85, 90, 95%, more preferably at least 100, 150, 200, 250, 300, 350 and 400%, even more preferably at least 450, 500, 550, 600% compared to liver cells with no treatment. In another embodiment, C/EBPα-saRNA decreases SREBP1 gene expression. In another embodiment, C/EBPα-saRNA decreases DGAT2 gene expression. In another embodiment, C/EBPα-saRNA decreases CETP gene expression. In yet another embodiment, C/EBPα-saRNA decreases FASN gene expression.

A summary of NAFLD and IR genes that may be modulated with C/EBPα-saRNA is shown in Table 4. Abbreviations in Table 4: NAFLD: non-alcoholic fatty liver disease; IR: insulin resistance; DNL: de novo lipogenesis; FA: fatty acid; TG: triglycerides; LPL: lipoprotein lipase; HP: hepatic lipase; CHOL: cholesterol.

TABLE 4
NAFLD and IR genes that may be modulated with C/EBPα-saRNA

| | | | Deregu- | Deregu- |
|---|---|---|---|---|
| Gene | | Function/encoded | lation in | lation |
| name | Mechanism | products - References | NAFLD | in IR |
| CD36 | FAs uptake | Scavenger receptor, free FAs | up | up |
| | | transporter in liver and adipose tissue; | | |
| | | regulates adipose tissue apoptosis | | |
| | | and inflammation | | |
| PPARγ | DNL | Activates genes involved in lipid | up | down |
| | | storage and metabolism; required for | | |
| | | lipid homeostasis; high expressed | | |
| | | in adipose tissue and very low in the | | |
| | | liver; implicated in adipocyte | | |
| | | differentiation and insulin sensitivity | | |
| PPARγ- | DNL | Transcriptional coactivator for | up | up |
| CoA 1β | | SREBP-1; enhances lipogenesis and | | |
| (PERC) | | VLDL synthesis; highly expressed in | | |
| | | brown fat and heart and induced in the | | |
| | | liver during fasting; master regulator of | | |
| | | mitochondrial biogenesis and oxidative | | |
| | | metabolism, lipogenesis, and TG | | |
| | | secretion | | |
| SREBP-1c | DNL | Transcription factor, induces genes | up | up |
| | | involved in glucose utilization and FA | | |
| | | synthesis; major mediator of insulin | | |
| | | action on lipogenic genes; | | |
| | | regulates adipogenesis | | |
| ChREBP | DNL | Transcription factors activated by | up | up |
| (MLX1PL) | | glucose; induces glycolytic and | | |
| | | lipogenic genes; major determinant of | | |
| | | adipose tissue fatty acid synthesis and | | |
| | | systemic insulin sensitivity | | |
| FAS | DNL | Enzyme that catalyzes the last step | up | up |
| | | in FA biosynthesis | | |
| ACACA | DNL | Enzyme that catalyzes the synthesis | up | up |
| (ACC1) | | of malonyl-CoA for the synthesis of | | |
| | | FAs in the cytosol | | |
| ACACB | β-oxidation | Enzyme that catalyzes the synthesis of | up | up |
| (ACC2) | | malonyl-CoA, which functions as | | |
| | | inhibitor of mitochondrial β-oxidation | | |
| PPARα | β-oxidation | Activates the genes involved in the | down | down |
| | | oxidation of FAs, major regulator of | | |
| | | lipid metabolism in the liver; | | |
| | | predominantly expressed in the liver; | | |
| | | involved in the regulation of glucose | | |
| | | homeostasis, insulin sensitivity, fat | | |
| | | accumulation, and adipose tissue | | |
| | | glucose use | | |
| PPARγ- | β-oxidation | Transcriptional co-activator that | down | down |
| CoA 1α | | regulates mitochondrial biology and | | |
| | | energy homeostasis; crucial role in | | |
| | | mitochondrial biogenesis; interacts | | |
| | | with PPARα to increase the | | |
| | | mitochondrial β-oxidation of FAs | | |
| DGAT2 | TG synthesis | Enzyme that catalyzes the final | up | up |
| | | reaction in the synthesis of TG | | |
| APOC3 | TG | Protein that inhibits LPL and HP; | up | up |
| | concentration | involved in the regulation of plasma | | |
| | | TG concentrations; pro-steatosic | | |
| LDLR | CHOL | Low-density lipoprotein receptor; | down | no |
| | concentration | critical role in regulating blood CHOL | | change |
| | | levels; abundant in the liver, which is | | |
| | | the organ responsible for removing | | |
| | | most excess CHOL from the body | | |
| MTP | Lipoprotein | Carrier of TG; central role in VLDL | down | no |
| (MTTP1) | assembly | assembly; prevalently expressed in | | change |
| | | the liver | | |
| mTOR | Adipose | Possible regulator of adipose tissue | up | up |
| | mass | mass; central role in lipolysis, | | |
| | | lipogenesis, and adipogenesis | | |

| | Effects of Ezetimibe in | Effects of C/EBPα | | |
|---|---|---|---|---|
| Gene name | the liver | Liver | WAT | BAT |
| CD36 | minor down | down | down | down |
| PPARγ | up | up | no change | no change |
| PPARγ-CoA | up | up | down | up |
| 1β (PERC) | | | | |
| SREBP-1c | up | down | down | down |
| ChREBP | up | down | up | up |
| (MLX1PL) | | | | |
| FAS | down | down | minor up | up |
| ACACA | minor up | no change | down | up |
| (ACC1) | | | | |
| ACACB | up | up | down | down |
| (ACC2) | | | | |
| PPARα | up | up | down | up |
| PPARγ-CoA | up | up | up | up |
| 1α | | | | |
| DGAT2 | minor down | minor down | down | up |
| APOC3 | down | down | up | down |
| LDLR | minor down | down | up | minor down |
| MTP (MTTP1) | up | down | up | down |
| mTOR | no change | no change | down | down |

In one embodiment of the present invention, provided is a method of lowering serum cholesterol level in vitro by treatment of C/EBPα-saRNA of the present invention. The serum cholesterol level with C/EBPα-saRNA reduces at least 25%, preferably 50%, more preferably 75% compared to serum cholesterol level with no treatment. Also provided is a method of lowering LDL and triglyceride levels in hepatocyte cells and increasing circulating levels of LDL in vivo by administering C/EBPα-saRNA of the present invention. The circulation LDL level may increase at least by a factor of 2, preferably by a factor of 3, preferably by a factor of 4, preferably by a factor of 5, preferably by a factor of 10, and preferably by a factor of 15 compared to circulating LDL level in the absence of C/EBPα-saRNA. The liver triglyceride level may be reduced by at least 10%, 20%, 30%, 40%, 50%, 60%, or 70% compared to the liver triglyceride level in the absence of C/EBPα-saRNA. The liver LDL level may be reduced by at least 10%, 20%, 30%, 40%, 50%, 60%, or 70% compared to the liver LDL level in the absence of C/EBPα-saRNA.

In one embodiment of the present invention, provided is a method of treating NAFLD and reducing fatty liver size by administering C/EBPα-saRNA of the present invention to a patient in need thereof. The size of a fatty liver of a patient treated with C/EBPα-saRNA is reduced by at least 10%, 20%, 30%, 40%, or 50% compared with a patient without treatment. Also provided is a method of reducing body weight and treating obesity by administering C/EBPα-saRNA of the present invention to a patient in need thereof. The body weight of a patient treated with C/EBPα-saRNA is lower than the body weight of a patient without treatment of C/EBPα-saRNA by at least 10%, 20%, 30%, 40%, 50%, 60%, or 70%. C/EBPα-saRNA of the present invention may be administered in a dose, 2 doses, 3 does or more. Also provided is a method of decreasing hepatic uptake of free fatty acids by treatment of C/EBPα-saRNA of the present invention. Also provided is a method of reducing white adipose tissue (WAT) inflammation by treatment of C/EBPα-saRNA of the present invention. Also provided is a method of reducing de novo lipogenesis by treatment of C/EBPα-saRNA of the present invention. Also provided is a method of increasing beta-oxidation in the liver by treatment of C/EBPα-saRNA of the present invention. Also provided is a method of increasing brown adipose tissue (BAT) in the liver by treatment of C/EBPα-saRNA of the present invention. Also provided is a method of reducing hepatic lipid uptake by treatment of C/EBPα-saRNA of the present invention. Also provided is a method of decreasing lipogenesis in WAT by treatment of C/EBPα-saRNA of the present invention. Also provided is a method of decreasing lipid storage in liver by treatment of C/EBPα-saRNA of the present invention. Also provided is a method of reducing lipid overload in the liver by treatment of C/EBPα-saRNA of the present invention.

In another embodiment, C/EBPα-saRNA of the present invention is used to increase liver function. In one non-limiting example, C/EBPα-saRNA increases albumin gene expression and thereby increasing serum albumin and unconjugated bilirubin levels. The expression of albumin gene may be increased by at least 20, 30, 40%, more preferably at least 45, 50, 55, 60, 65, 70, 75%, even more preferably at least 80% in the presence of the saRNA of the present invention compared to the expression of albumin gene in the absence of the saRNA of the present invention. In a further preferable embodiment, the expression of albumin gene is increased by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of the saRNA of the present invention compared to the expression of albumin gene in the absence of the saRNA of the present invention. In another non-limiting example, C/EBPα-saRNA decreases the amount of alanine transaminase (ALT), aspartate aminotransferase (AST), gamma glutamyl transpeptidase (GGT), alphafectoprotein (AFP) and hepatocyte growth factor (HGF). The amount of ALT, AST, GGT, AFP, or HGF may be decreased by at least 20, 30, 40%, more preferably at least 45, 50, 55, 60, 65, 70, 75%, even more preferably at least 80% in the presence of the saRNA of the present invention compared to the amount of any of ALT, AST, GGT, AFP, or HGF in the absence of the saRNA of the present invention.

In another embodiment, C/EBPα-saRNA of the present invention is administered to regulate the levels of other members of the C/EBP family. C/EBPα-saRNA increases the expression of C/EBPβ, C/EBPγ, C/EBPδ and C/EBPζ depending on the dose of C/EBPα-saRNA. In yet another embodiment, the ratio of C/EBPα or C/EBPβ protein isoforms in a cell is regulated by contacting said cell with C/EBPα-saRNA of the present invention. In one embodiment, the 42 KDa isoform of C/EBPα is increased. In one embodiment, the 30 kDa isoform of C/EBPβ is increased.

### Surgical Care

Hepatectomy, surgical resection of the liver or hepatic tissue might cause liver failure, reduced production of albumin and coagulation factors. Proper surgical care after hepatectomy is needed. In some embodiments, C/EBPα-saRNA of the present invention is used for surgical care after hepatectomy to promote liver regeneration and increase survival rate.

### Hyperproliferation Disorders

In one embodiment of the invention, C/EBPα-saRNA of the present invention is used to reduce cell proliferation of hyperproliferative cells. Examples of hyperproliferative cells include cancerous cells, e.g., carcinomas, sarcomas, lymphomas and blastomas. Such cancerous cells may be benign or malignant. Hyperproliferative cells may result from an autoimmune condition such as rheumatoid arthritis, inflammatory bowel disease, or psoriasis. Hyperproliferative cells may also result within patients with an oversensitive immune system coming into contact with an allergen. Such conditions involving an oversensitive immune system include, but are not limited to, asthma, allergic rhinitis, eczema, and allergic reactions, such as allergic anaphylaxis. In one embodiment, tumor cell development and/or growth is inhibited. In a preferred embodiment, solid tumor cell proliferation is inhibited. In another preferred embodiment, metastasis of tumor cells is prevented. In another preferred example, undifferentiated tumor cell proliferation is inhibited.

Inhibition of cell proliferation or reducing proliferation means that proliferation is reduced or stops altogether. Thus, “reducing proliferation” is an embodiment of “inhibiting proliferation”. Proliferation of a cell is reduced by at least 20%, 30% or 40%, or preferably at least 45, 50, 55, 60, 65, 70 or 75%, even more preferably at least 80, 90 or 95% in the presence of the saRNA of the invention compared to the proliferation of said cell prior to treatment with the saRNA of the invention, or compared to the proliferation of an equivalent untreated cell. In embodiments wherein cell proliferation is inhibited in hyperproliferative cells, the “equivalent” cell is also a hyperproliferative cell. In preferred embodiments, proliferation is reduced to a rate comparable to the proliferative rate of the equivalent healthy (non-hyperproliferative) cell. Alternatively viewed, a preferred embodiment of “inhibiting cell proliferation” is the inhibition of hyperproliferation or modulating cell proliferation to reach a normal, healthy level of proliferation.

In one non-limiting example, C/EBPα-saRNA is used to reduce the proliferation of leukemia and lymphoma cells. Preferably, the cells include Jurkat cells (acute T cell lymphoma cell line), K562 cells (erythroleukemia cell line), U373 cells (glioblastoma cell line), and 32Dp210 cells (myeloid leukemia cell line).

In another non-limiting example, C/EBPα-saRNA is used to reduce the proliferation of ovarian cancer cells, liver cancer cells, pancreatic cancer cells, breast cancer cells, prostate cancer cells, rat liver cancer cells, and insulinoma cells. Preferably, the cells include PEO1 and PEO4 (ovarian cancer cell line), HepG2 (hepatocellular carcinoma cell line), Pancl (human pancreatic carcinoma cell line), MCF7 (human breast adenocarcinoma cell line), DU145 (human metastatic prostate cancer cell line), rat liver cancer cells, and MIN6 (rat insulinoma cell line).

In another non-limiting example, C/EBPα-saRNA is used in combination with a siRNA targeting C/EBβ gene to reduce tumor cell proliferation. Tumor cell may include hepatocellular carcinoma cells such as HepG2 cells and breast cancer cells such as MCF7 cells.

In one embodiment, the saRNA of the present invention is used to treat hyperproliferative disorders. Tumors and cancers represent a hyperproliferative disorder of particular interest, and all types of tumors and cancers, e.g. solid tumors and haematological cancers are included. Examples of cancer include, but not limited to, cervical cancer, uterine cancer, ovarian cancer, kidney cancer, gallbladder cancer, liver cancer, head and neck cancer, squamous cell carcinoma, gastrointestinal cancer, breast cancer, prostate cancer, testicular cancer, lung cancer, non-small cell lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer (e.g. astrocytoma, glioblastoma, medulloblastoma), neuroblastoma, sarcomas, colon cancer, rectum cancer, stomach cancer, anal cancer, bladder cancer, endometrial cancer, plasmacytoma, lymphomas, retinoblastoma, Wilm's tumor, Ewing sarcoma, melanoma and other skin cancers. The liver cancer may include, but not limited to, cholangiocarcinoma, hepatoblastoma, haemangiosarcoma, or hepatocellular carcinoma (HCC). HCC is of particular interest.

Primary liver cancer is the fifth most frequent cancer worldwide and the third most common cause of cancer-related mortality. HCC represents the vast majority of primary liver cancers [El-Serag et al., Gastroenterology, vol. 132(7), 2557-2576 (2007), the contents of which are disclosed herein in their entirety]. HCC is influenced by the interaction of several factors involving cancer cell biology, immune system, and different aetiologies (viral, toxic and generic). The majority of patients with HCC develop malignant tumors from a background of liver cirrhosis. Currently most patients are diagnosed at an advanced stage and therefore the 5 year survival for the majority of HCC patients remains dismal. Surgical resection, loco-regional ablation and liver transplantation are currently the only therapeutic options which have the potential to cure HCC. However, based on the evaluation of individual liver function and tumor burden only about 5-15% of patients are eligible for surgical intervention. The binding sites for the family of C/EBP transcription factors are present in the promoter regions of numerous genes that are involved in the maintenance of normal hepatocyte function and response to injury (including albumin, interleukin 6 response, energy homeostasis, ornithine cycle regulation and serum amyloid A expression). The present invention utilizes C/EBPα-saRNA to modulate the expression of C/EBPα gene and treat liver cirrhosis and HCC.

The method of the present invention may reduce tumor volume by at least 10, 20, 30, 40, 50, 60, 70, 80 or 90%. Preferably, the development of one or more new tumors is inhibited, e.g. a subject treated according to the invention develops fewer and/or smaller tumors. Fewer tumors means that he develops a smaller number of tumors than an equivalent subject over a set period of time. For example, he develops at least 1, 2, 3, 4 or 5 fewer tumors than an equivalent control (untreated) subject. Smaller tumor means that the tumors are at least 10, 20, 30, 40, 50, 60, 70, 80 or 90% smaller in weight and/or volume than tumors of an equivalent subject. The method of the present invention reduces tumor burden by at least 10, 20, 30, 40, 50, 60, 70, 80 or 90%.

The set period of time may be any suitable period, e.g. 1, 2, 3, 4, 5, 6, 7, 8, 9 or 10 months or years.

In one non-limiting example, provided is a method of treating an undifferentiated tumor, comprising contacting a cell, tissue, organ or subject with C/EBPα-saRNA of the present invention. Undifferentiated tumors generally have a poorer prognosis compared to differentiated ones. As the degree of differentiation in tumors has a bearing on prognosis, it is hypothesized that the use of a differentiating biological agent could be a beneficial anti-proliferative drug. C/EBPα is known to restore myeloid differentiation and prevent hyperproliferation of hematopoietic cells in acute myeloid leukemia. Preferably, undifferentiated tumors that may be treated with C/EBPα-saRNA include undifferentiated small cell lung carcinomas, undifferentiated pancreatic adenocarcinomas, undifferentiated human pancreatic carcinoma, undifferentiated human metastatic prostate cancer, and undifferentiated human breast cancer.

In one non-limiting example, C/EBPα-saRNA is complexed into PAMAM dendrimer, referred to as C/EBPα-saRNA-dendrimer for targeted in vivo delivery. The therapeutic effect of intravenously injected C/EBPα-saRNA-dendrimers is demonstrated in a clinically relevant rat liver tumor model as shown in Example 1. After three doses through tail vein injection at 48 hour intervals, the treated cirrhotic rats showed significantly increased serum albumin levels within one week. The liver tumor burden was significantly decreased in the C/EBPα-saRNA dendrimer treated groups. This study demonstrates, for the first time, that gene targeting by small activating RNA molecules can be used by systemic intravenous administration to simultaneously ameliorate liver function and reduce tumor burden in cirrhotic rats with HCC.

In one embodiment, C/EBPα-saRNA is used to regulate oncogenes and tumor suppressor genes. Preferably, the expression of the oncogenes may be down-regulated. The expression of the oncogenes reduces by at least 20, 30, 40%, more preferably at least 45, 50, 55, 60, 65, 70, 75, 80, 85, 90, 95% in the presence of C/EBPα-saRNA of the invention compared to the expression in the absence of C/EBPα-saRNA of the invention. In a further preferable embodiment, the expression of the oncogenes is reduced by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of C/EBPα-saRNA of the invention compared to the expression in the absence of C/EBPα-saRNA of the invention. Preferably, the expressions of tumor suppressor genes may be inhibited. The expression of the tumor suppressor genes increase by at least 20, 30, 40%, more preferably at least 45, 50, 55, 60, 65, 70, 75, 80, 85,90, 95%, even more preferably at least 100% in the presence of C/EBPα-saRNA of the invention compared to the expression in the absence of C/EBPα-saRNA of the invention. In a further preferable embodiment, the expression of tumor suppressor genes is increased by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100 in the presence of C/EBPα-saRNA of the invention compared to the expression in the absence of C/EBPα-saRNA of the invention. Non-limiting examples of oncogenes and tumor suppressor genes include Bcl-2-associated X protein (BAX), BH3 interacting domain death agonist (BID), caspase 8 (CASP8), disabled homolog 2-interacting protein (DAB21P), deleted in liver cancer 1 (DLC1), Fas surface death receptor (FAS), fragile histidine triad (FHIT), growth arrest and DNA-damage-inducible-beta (GADD45B), hedgehog interacting protein (HHIP), insulin-like growth factor 2 (IGF2), lymphoid enhancer-binding factor 1 (LEF1), phosphatase and tensin homolog (PTEN), protein tyrosine kinase 2 (PTK2), retinoblastoma 1 (RB1), runt-related transcription factor 3 (RUNX3), SMAD family member 4 (SMAD4), suppressor of cytokine signaling (3SOCS3), transforming growth factor, beta receptor II (TGFBR2), tumor necrosis factor (ligand) superfamily, member 10 (TNFSF10), P53, disintegrin and metalloproteinase domain-containing protein 17(ADAM17), v-akt murine thymoma viral oncogene homolog 1 (AKT1), angiopoietin 2 (ANGPT2), B-cell CLL/lymphoma 2 (BCL2), BCL2-like 1 (BCL2L1), baculoviral IAP repeat containing 2 (BIRC2), baculoviral IAP repeat containing 5 (BIRC5), chemokine (C-C motif) ligand 5 (CCL5), cyclin D1 (CCND1), cyclin D2 (CCND2), cadherin 1 (CDH1), cadherin 13 (CDH13), cyclin-dependent kinase inhibitor 1A (CDKN1A), cyclin-dependent kinase inhibitor 1B (CDKN1B), cyclin-dependent kinase inhibitor 2A (CDKN2A), CASP8 and FADD-like apoptosis regulator (CFLAR), catenin (cadherin-associated protein) beta 1 (CTNNB1), chemokine receptor 4 (CXCR4), E2F transcription factor 1 (E2F1), epidermal growth factor (EGF), epidermal growth factor receptor (EGFR), E1A binding protein p300 (EP300), Fas (TNFRSF6)-associated via death domain (FADD), fms-related tyrosine kinase 1 (FLT1), frizzled family receptor 7 (FZD7), glutathione S-transferase pi 1 (GSTP1), hepatocyte growth factor (HGF), Harvey rat sarcoma viral oncogene homolog (HRAS), insulin-like growth factor binding protein 1 (IGFBP1), insulin-like growth factor binding protein 3 (IGFBP3), insulin receptor substrate 1 (IRS1), integrin beta 1 (ITGB1), kinase insert domain receptor (KDR), myeloid cell leukemia sequence 1 (MCL1), met proto-oncogene (MET), mutS homolog 2 (MSH2), mutS homolog 3 (MSH3), metadherin (MTDH), v-myc avian myelocytomatosis viral oncogene homolog (MYC), nuclear factor of kappa light polypeptide gene enhancer in B-cells 1 (NFKB1), neuroblastoma RAS viral (v-ras) oncogene homolog (NRAS), opioid binding protein/cell adhesion molecule-like (OPCML), platelet-derived growth factor receptor, alpha polypeptide (PDGFRA), peptidylprolyl cis/trans isomerase, NIMA-interacting 1 (PIN1), prostaglandin-endoperoxide synthase 2 (PTGS2), PYD and CARD domain containing (PYCARD), ras-related C3 botulinum toxin substrate 1 (RAC1), Ras association (RalGDS/AF-6) domain family member 1 (RASSF1), reelin (RELN), ras homolog family member A (RHOA), secreted frizzled-related protein 2 (SFRP2), SMAD family member 7 (SMAD7), suppressor of cytokine signaling 1 (SOCS1), signal transducer and activator of transcription 3 (STAT3), transcription factor 4 (TCF4), telomerase reverse transcriptase (TERT), transforming growth factor alpha (TGFA), transforming growth factor beta 1 (TGFB1), toll-like receptor 4 (TLR4), tumor necrosis factor receptor superfamily member 10b (TNFRSF10B), vascular endothelial growth factor A (VEGFA), Wilms tumor 1 (WT1), X-linked inhibitor of apoptosis (XIAP), and Yes-associated protein 1 (YAP1).

In one embodiment, provided is a method of increasing white blood cell count by administering C/EBPα-saRNA of the present invention to a patient in need thereof. Also provided is a method of treating leukopaenia for patients having sepsis or chronic inflammation diseases (e.g., hepatitis and liver cirrhosis) and for immunocompromised patients (e.g., patients undergoing chemotherapy) by administering C/EBPα-saRNA of the present invention to said patient. Also provided is a method of treating pre B cell and B cell malignancies including leukaemia and lymphoma by administering C/EBPα-saRNA of the present invention to a patient in need thereof. Also provided is a method of mobilize white blood cells, haematopoietic or mesenchymal stem cells by administering C/EBPα-saRNA of the present invention to a patient in need thereof In one embodiment, the white blood cell count in a patient treated with C/EBPα-saRNA is increased by at least 50%, 75%, 100%, more preferably by at least a factor of 1.5, 2, 2.5, 3, 3.5, 4, 4.5, 5, more preferably by at least a factor of 6, 7, 8, 9, 10 compared to no C/EBPα-saRNA treatment.

In one embodiment, C/EBPα-saRNA is used to regulate micro RNAs (miRNA or miR) in the treatment of hepatocellular carcinoma. MicroRNAs are small non-coding RNAs that regulate gene expression. They are implicated in important physiological functions and they may be involved in every single step of carcinogenesis. They typically have 21 nucleotides and regulate gene expression at the post transcriptional level via blockage of mRNA translation or induction of mRNA degradation by binding to the 3′-untranslated regions (3′-UTR) of said mRNA.

In tumors, regulation of miRNA expression affects tumor development. In HCC, as in other cancers, miRNAs function either as oncogenes or tumor suppressor genes influencing cell growth and proliferation, cell metabolism and differentiation, apoptosis, angiogenesis, metastasis and eventually prognosis. [Lin et al., Biochemical and Biophysical Research Communications, vol. 375, 315-320 (2008); Kutay et al., J. Cell. Biochem., vol. 99, 671-678 (2006); Meng et al., Gastroenterology, vol. 133(2), 647-658 (2007), the contents of each of which are incorporated herein by reference in their entirety] C/EBPα-saRNA of the present invention modulates C/EBPα gene expression and/or function and also regulates miRNA levels in HCC cells. Non-limiting examples of miRNAs that may be regulated by C/EBPα-saRNA of the present invention include hsa-let-7a-5p, hsa-miR-133b, hsa-miR-122-5p, hsa-miR-335-5p, hsa-miR-196a-5p, hsa-miR-142-5p, hsa-miR-96-5p, hsa-miR-184, hsa-miR-214-3p, hsa-miR-15a-5p, hsa-let-7b-5p, hsa-miR-205-5p, hsa-miR-181a-5p, hsa-miR-140-5p, hsa-miR-146b-5p, hsa-miR-34c-5p, hsa-miR-134, hsa-let-7g-5p, hsa-let-7c, hsa-miR-218-5p, hsa-miR-206, hsa-miR-124-3p, hsa-miR-100-5p, hsa-miR-10b-5p, hsa-miR-155-5p, hsa-miR-1, hsa-miR-150-5p, hsa-let-7i-5p, hsa-miR-27b-3p, hsa-miR-12′7-5p, hsa-miR-191-5p, hsa-let-7f-5p, hsa-miR-10a-5p, hsa-miR-15b-5p, hsa-miR-16-5p, hsa-miR-34a-5p, hsa-miR-144-3p, hsa-miR-128, hsa-miR-215, hsa-miR-193a-5p, hsa-miR-23b-3p, hsa-miR-203a, hsa-miR-30c-5p, hsa-let-7e-5p, hsa-miR-146a-5p, hsa-let-7d-5p, hsa-miR-9-5p, hsa-miR-181b-5p, hsa-miR-181c-5p, hsa-miR-20b-5p, hsa-miR-125a-5p, hsa-miR-148b-3p, hsa-miR-92a-3p, hsa-miR-378a-3p, hsa-miR-130a-3p, hsa-miR-20a-5p, hsa-miR-132-3p, hsa-miR-193b-3p, hsa-miR-183-5p, hsa-miR-148a-3p, hsa-miR-138-5p, hsa-miR-3′73-3p, hsa-miR-29b-3p, hsa-miR-135b-5p, hsa-miR-21-5p, hsa-miR-181d, hsa-miR-301a-3p, hsa-miR-200c-3p, hsa-miR-7-5p, hsa-miR-29a-3p, hsa-miR-210, hsa-miR-17-5p, hsa-miR-98-5p, hsa-miR-25-3p, hsa-miR-143-3p, hsa-miR-19a-3p, hsa-miR-18a-5p, hsa-miR-125b-5p, hsa-miR-126-3p, hsa-miR-27a-3p, hsa-miR-372, hsa-miR-149-5p, and hsa-miR-32-5p.

In one non-limiting example, the miRNAs are oncogenic miRNAs and are downregulated by a factor of at least 0.01, 0.02, 0.05, 0.1, 0.2, 0.3, 0.5, 1, 1.5, 2, 2.5, and 3, in the presence of C/EBPα-saRNA of the invention compared to in the absence of C/EBPα-saRNA. In another non-limiting example, the miRNAs are tumor suppressing miRNAs and are upregulated by a factor of at least 0.01, 0.02, 0.05, 0.1, 0.2, 0.3, 0.5, 1, more preferably by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of C/EBPα-saRNA of the invention compared to in the absence of C/EBPα-saRNA.

Combination with Other Therapies

The saRNA of the present invention may be provided in combination with additional active agents or therapies known to have an effect in the particular method being considered. For example, the combination therapy comprising saRNA and additional active agents or therapies may be given to any patient in need thereof to treat any disorder described herein, including metabolics regulation, surgical care, hyperproliferative disorders, and/or stem cell regulation.

The additional active agents may be administered simultaneously or sequentially with the saRNA. The additional active agents may be administered in a mixture with the saRNA or be administered separately from the saRNA.

The term “administered simultaneously” as used herein is not specifically restricted and means that the components of the combination therapy, i.e., saRNA of the present invention and the additional active agents, are substantially administered at the same time, e.g. as a mixture or in immediate subsequent sequence.

The term “administered sequentially” as used herein is not specifically restricted and means that the components of the combination therapy, i.e., saRNA of the present invention and the additional active agents, are not administered at the same time but one after the other, or in groups, with a specific time interval between administrations. The time interval may be the same or different between the respective administrations of the components of the combination therapy and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours. In some embodiments, the saRNA of the present invention is administered before the additional active agents. In some embodiments, the additional active agents are administered before the saRNA of the present invention.

The molar ratio of the saRNA of the present invention and the additional active agents is not particularly restricted. For example, when two components are combined in a composition, the molar ratio between the two components may be in the range of 1:500 to 500:1, or of 1:100 to 100:1, or of 1:50 to 50:1, or of 1:20 to 20:1, or of 1:5 to 5:1, or 1:1. Similar molar ratios apply when more than two components are combined in a composition. Each component may comprise, independently, a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

In one embodiment, C/EBPα-saRNA is administered with saRNA modulating a different target gene. Non-limiting examples include saRNA that modulates albumin, insulin or HNF4A genes. Modulating any gene may be achieved using a single saRNA or a combination of two or more different saRNAs. Non-limiting examples of saRNA that can be administered with C/EBPα-saRNA of the present invention include saRNA modulating albumin or HNF4A disclosed in International Publication WO 2012/175958 filed Jun. 20, 2012, saRNA modulating insulin disclosed in International Publications WO 2012/046084 and WO 2012/046085 both filed Oct. 10, 2011, saRNA modulating human progesterone receptor, human major vault protein (hMVP), E-cadherin gene, p53 gene, or PTEN gene disclosed in U.S. Pat. No. 7,709,456 filed Nov. 13, 2006 and US Pat. Publication US 2010/0273863 filed Apr. 23, 2010, and saRNAs targeting p21 gene disclosed in International Publication WO 2006/113246 filed Apr. 11, 2006, the contents of each of which are incorporated herein by reference in their entirety.

In one embodiment, C/EBPα-saRNA is administered in combination with a small interfering RNA or siRNA that inhibits the expression of C/EBPβ gene, i.e., C/EBPβ-siRNA.

In one embodiment, C/EBPα-saRNA is administered with one or more drugs that regulate metabolics, particularly liver function. In a non-limiting example, C/EBPα-saRNA of the present invention is administered with drugs that decrease low density lipoprotein (LDL) cholesterol levels, such as statin, simvastatin, atorvastatin, rosuvastatin, ezetimibe, niacin, PCSK9 inhibitors, CETP inhibitors, clofibrate, fenofibric, tocotrienols, phytosterols, bile acid sequestrants, probucol, or a combination thereof. C/EBPα-saRNA may also be administered with vanadium biguanide complexes disclosed in U.S. Pat. No. 6,287,586 to Orvig et al. In another example, C/EBPα-saRNA may be administered with a composition disclosed in WO 201102838 to Rhodes, the contents of which are incorporated by reference in their entirety, to lower serum cholesterol. The composition comprises an antigen binding protein that selectively binds to and inhibits a PCSK9 protein; and an RNA effector agent which inhibits the expression of a PCSK9 gene in a cell. In yet another example, C/EBPα-saRNA may be administered with an ABC1 polypeptide having ABC1 biological activity, or a nucleic acid encoding an ABC1 polypeptide having ABC1 activity to modulate cholesterol levels as described in EP1854880 to Brooks-Wilson et al., the contents of which are incorporated herein by reference in their entirety.

In another embodiment, C/EBPα-saRNA of the present invention is administered with drugs that increase insulin sensitivity or treat type II diabetes mellitus, such as metformin, sulfonylurea, nonsulfonylurea secretagogues, a glucosidase inhibitors, thiazolidinediones, pioglitazone, rosiglitazone, glucagon-like peptide-1 analog, and dipeptidyl peptidase-4 inhibitors or a combination thereof. Other hepato-protective agents that may be administered in combination with the saRNA of the present invention are disclosed in Adams et al., Postgraduate Medical Journal, vol. 82, 315-322 (2006), the contents of which are incorporated herein by reference in their entirety.

FGFR4 Inhibitors

Fibroblast Growth Factor Receptor 4 (FGFR4) gene encodes FGFR4 protein, which is a tyrosine kinase and a cell surface receptor for fibroblast growth factors. FGFR4 protein regulates pathways involved in cell proliferation, differentiation, and migration; lipid metabolism; bile acid biosynthesis; glucose uptake; and phosphate homeostasis. Aberrant signaling through the fibroblast growth factor 19 (FGF19)/FGFR4 signaling complex has been shown to involve in hepatocellular carcinoma (HCC) in mice and may play a similar role in humans.

C/EBPα-saRNA of the present invention may be used in combination with one or more of therapeutic agents that down-regulate FGFR4 levels or inhibit FGFR4 receptor signaling. The combination may have synergistic effect on preventing and/or treating any cancer, such as but not limited to HCC. The therapeutic agent that down-regulate FGFR4 levels or inhibit FGFR4 signaling may be an FGFR4 inhibitor.

In some embodiments, the FGFR4 inhibitor is a small inhibiting RNA (FGFR4-siRNA) that reduce the expression of the FGFR4 gene. The siRNA may be single stranded or double stranded. Non-limiting examples of FGFR4-siRNAs include siRNA s5176 (ThermoFisher Scientific).

In some embodiments, the FGFR4 inhibitor is an FGFR4 antagonist antibody. Non-limiting examples of FGFR4 antibodies include U3-1784.

In some embodiments, the FGFR4 inhibitor is a small molecule inhibitor. Non-limiting examples of small molecule FGFR4 inhibitors include BGJ398 (Novartis), H3B-6527 (H3 Biomedicine), BLU-9931 (BluePrint Medicines), and BLU-554 (BluePrint Medicines).

In some embodiments, the patients receiving a combination therapy of C/EBPα-saRNA and at least one FGFR4 inhibitor may have HCC. The patients may be treated with an FGFR4 inhibitor first, followed by a treatment with C/EBPα-saRNA; be treated with C/EBPα-saRNA first, followed by a treatment with an FGFR4 inhibitor; or be treated with a composition comprising both C/EBPα-saRNA and an FGFR4 inhibitor.

C/EBPβ Inhibitors

C/EBPβ (or CEBPB) promotes tumorigenesis by modulating the expression of genes encoding cytokines and chemokines, and by regulating cell cycle progression and apoptosis. C/EBPβ knockdown has previously been shown to activate CEBPA expression by stimulating the expression of the transcription factor peroxisome proliferator-activated receptor gamma (PPARγ) and dislodging histone deacetylase 1 (HDAC1) from the CEBPA promoter (Zuo et al., Journal of Biological Chemistry, vol.281:7960 (2006)). There is a dynamic interaction between C/EBPα and C/EBPβ during liver regeneration. A high ratio of C/EBPα to C/EBPβ suppresses cell proliferation by repressing cell cycle and acute phase response genes and activating metabolic genes, whereas a low ratio of C/EBPα to C/EBPβ has an opposite effect. However, the roles of these transcription factors as potential tools for regulating liver tumour development remain unknown.

C/EBPα-saRNA of the present invention may be used in combination with one or more of therapeutic agents that down-regulate C/EBPβ levels. The combination may have synergistic effect on preventing and/or treating any cancer, such as but not limited to HCC. The therapeutic agent that down-regulate C/EBPβ levels may be a C/EBPβ inhibitor.

In some embodiments, the C/EBPβ inhibitor is a small inhibiting RNA (C/EBPβ-siRNA) that reduce the expression of the C/EBPβ gene. The siRNA may be single stranded or double stranded.

In some embodiments, the C/EBPβ inhibitor is a C/EBPβ antagonist antibody.

In some embodiments, the C/EBPβ inhibitor is a small molecule inhibitor.

In some embodiments, the patients receiving a combination therapy of C/EBPα-saRNA and at least one C/EBPβ inhibitor may have HCC. The patients may be treated with a C/EBPβ inhibitor first, followed by a treatment with C/EBPα-saRNA; be treated with C/EBPα-saRNA first, followed by a treatment with a C/EBPβ inhibitor; or be treated with a composition comprising both C/EBPα-saRNA and a C/EBPβ inhibitor.

Immunotherapies

In some embodiments, the C/EBPα-saRNA and/or compositions of the present application may be combined with another therapy, such as surgical treatment, radiation therapy, immunotherapy, gene therapy, and/or with any other antineoplastic treatment method.

As used herein, the term “immunotherapy” refers to any therapy that can provoke and/or enhance an immune response to destroy tumor cells in a subject.

In some embodiments, the C/EBPα-saRNA and/or compositions of the present application may be combined with cancer vaccines and/or complementary immunotherapeutics such as immune checkpoint inhibitors. As used herein, the term “vaccine” refers to a composition for generating immunity for the prophylaxis and/or treatment of diseases.

In some embodiments, the checkpoint inhibitor may be an antagonist agent against CTLA-4 such as an antibody, a functional fragment of the antibody, a polypeptide, or a functional fragment of the polypeptide, or a peptide, which can bind to CTLA-4 with high affinity and prevent the interaction of B7-1/2 (CD80/86) with CTLA-4. In one example, the CTLA-4 antagonist is an antagonistic antibody, or a functional fragment thereof. Suitable anti-CTLA-4 antagonistic antibody include, without limitation, anti-CTLA-4 antibodies, human anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and the antibodies disclosed in U.S. Pat. Nos.: 8,748,815; 8,529,902; 8,318,916; 8,017,114; 7,744,875; 7,605,238; 7,465,446; 7,109,003; 7,132,281; 6,984,720; 6,682,736; 6,207,156; 5,977,318; and European Patent No. EP1212422B1; and U.S. Publication Nos. US 2002/0039581 and US 2002/086014; and Hurwitz et al., Proc. Natl. Acad. Sci. USA, 1998, 95(17):10067-10071; the contents of each of which are incorporated by reference herein in their entirety.

Additional anti-CTLA-4 antagonist agents include, but are not limited to, any inhibitors that are capable of disrupting the ability of CTLA-4 to bind to the ligands CD80/86.

In some embodiments, the checkpoint inhibitor may be agents used for blocking the PD-1 pathway include antagonistic peptides/antibodies and soluble PD-L1 ligands (See Table 5).

TABLE 5
Agents that block the inhibitory PD-1 and PD-L1 pathway

| Agent | Description | Target |
|---|---|---|
| Nivolumab | Human IgG | PD-1 |
| (BMS-936558, ONO-4538, | | |
| MDX-1106 | | |
| Pembrolizumab | Humanized IgG4 | PD-1 |
| (MK-3475, lambrolizumab, | | |
| Keytruda ®) | | |
| Pidilizumab (CT-011) | Humanized anti-PD-1 | PD-1 |
| | IgG1kappa | |
| AMP-224 | B7-DC/IgG1 fusion protein | PD-1 |
| MSB0010718 (EMD-Serono) | Human IgG1 | PD-L1 |
| MEDI4736 | Engineered human IgG | PD-L1 |
| | 1kappa | |
| MPDL3280A | Engineered IgG1 | PD-L1 |
| AUNP-12 | branched 29-amino acid | PD-1 |
| | peptide | |

In some embodiments, the C/EBPα-saRNA and/or compositions of the present application may be combined with a gene therapy, such as CRISPR (Clustered Regularly Interspaced Short Palidromic Repeats) therapy. As used herein, CRISPR therapy refers to any treatment that involves CRISPR-Cas system for gene editing.

In some embodiments, C/EBPα-saRNA of the present invention may be used in combination with one or more immune checkpoint blockade (ICB) agent. The combination may have synergistic effect on preventing and/or treating any cancer, such as but not limited to HCC.

In some embodiments, the ICB is a small inhibiting RNA (siRNA). The siRNA may be single stranded or double stranded.

In some embodiments, the ICB is an antibody.

In some embodiments, the ICB is a small molecule.

In some embodiments, the ICB is any agent in checkpoint inhibitor in Table 5.

In some embodiments, the ICB is Pembroluzimab, Tremelimumab, Durvalumab or Nivolumab.

In some embodiments, the patients receiving a combination therapy of C/EBPα-saRNA and at least one ICB may have HCC. The patients may be treated with an ICB first, followed by a treatment with C/EBPα-saRNA; be treated with C/EBPα-saRNA first, followed by a treatment with an ICB; or be treated with a composition comprising both C/EBPα-saRNA and ICB.

Radiofrequency Ablation (RFA)

Radiofrequency ablation (RFA) is the process by which tumour is destroyed using heat, generated by a high frequency alternating current and applied through an electrode tip. RFA is one of the standard treatment options for HCC in clinical practice and is associated with a significant survival benefit. Following RFA, the localised coagulation necrosis of the tumour remains in the body and provides proinflammatory signals to induce the release of large amounts of cellular debris that represents a source of tumour antigens which can trigger a host adaptive immune response against the tumour. Evidence suggests that tumour thermal ablation induces modulation of both innate and adaptive immune systems, inducing anti-tumour immune responses through efficient loading of dendritic cells, enhanced antigen presentation and an amplified tumour-specific T-cell response.

In some embodiments, C/EBPα-saRNA of the present invention may be used in combination with RFA process. A patient may receive RFA before, during, or after C/EBPα-saRNA treatments. A patient may further receive an immunotherapy, such as PD-1 inhibitor treatments.

Tyrosine Kinase Inhibitors (TKI)

Not willing to be bound by any theory, loss of function of C/EBP-a resulted in an increase in Myeloid Derived Suppressor Cells (MDSCs) in the tumour immune microenvironment resulting in augmented tumour growth in mouse models of cancer. MDSCs have been identified as key players in promoting a range of diseases, including in cancer where MDSCs may provide tumors resistance to cancer therapies. C/EBPα-saRNA of the present invention may be used to improve efficacy of various cancer therapies, such as tyrosine kinase inhibitors (TKI).

In some embodiments, C/EBPα-saRNA of the present invention may be used in combination with one or more tyrosine kinase inhibitors. TKIs are effective in the targeted treatment of various malignancies. Non-limiting example of tyrosine kinase inhibitors include imatinib, gefitinib, erlotinib, sorafenib, sunitinib, dasatinib, and lenvatinib.

In some embodiments, at least one TKI is administered after treatment with C/EBPα-saRNA of the present invention.

In some embodiments, at least one TKI is administered concomitantly with C/EBPα-saRNA of the present invention.

### III. Kits and Devices

### Kits

The invention provides a variety of kits for conveniently and/or effectively carrying out methods of the present invention. Typically, kits will comprise sufficient amounts and/or numbers of components to allow a user to perform multiple treatments of a subject(s) and/or to perform multiple experiments.

In one embodiment, the kits comprising saRNA described herein may be used with proliferating cells to show efficacy.

In one embodiment, the present invention provides kits for regulate the expression of genes in vitro or in vivo, comprising C/EBPα-saRNA of the present invention or a combination of C/EBPα-saRNA, saRNA modulating other genes, siRNAs, or miRNAs. The kit may further comprise packaging and instructions and/or a delivery agent to form a formulation composition. The delivery agent may comprise a saline, a buffered solution, a lipidoid, a dendrimer or any delivery agent disclosed herein. Non-limiting examples of genes include C/EBPα, other members of C/EBP family, albumin gene, alphafectoprotein gene, liver specific factor genes, growth factors, nuclear factor genes, tumor suppressing genes, pluripotency factor genes.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046; herein incorporated by reference in its entirety). In yet another non-limiting example, the buffer solutions may be precipitated, or it may be lyophilized. The amount of each component may be varied to enable consistent, reproducible higher concentration saline or simple buffer formulations. The components may also be varied in order to increase the stability of saRNA in the buffer solution over a period of time and/or under a variety of conditions.

In another embodiment, the present invention provides kits to regulate the proliferation of cells, comprising C/EBPα-saRNA of the present invention, provided in an amount effective to inhibit the proliferation of cells when introduced into said cells; optionally siRNAs and miRNAs to further regulate the proliferation of target cells; and packaging and instructions and/or a delivery agent to form a formulation composition.

In another embodiment, the present invention provides kits for reducing LDL levels in cells, comprising saRNA molecules of the present invention; optionally LDL reducing drugs; and packaging and instructions and/or a delivery agent to form a formulation composition.

In another embodiment, the present invention provides kits for regulating miRNA expression levels in cells, comprising C/EBPα-saRNA of the present invention; optionally siRNAs, eRNAs and lncRNAs; and packaging and instructions and/or a delivery agent to form a formulation composition.

In another embodiment, the present invention provides kits for combinational therapies comprising C/EBPα-saRNA of the present invention and at least one other active ingredient or therapy.

### Devices

The present invention provides for devices which may incorporate C/EBPα-saRNA of the present invention. These devices contain in a stable formulation available to be immediately delivered to a subject in need thereof, such as a human patient. Non-limiting examples of such a subject include a subject with hyperproliferative disorders such as cancer, tumor, or liver cirrhosis; and metabolics disorders such as NAFLD, obesity, high LDL cholesterol, or type II diabetes.

In some embodiments, the device contains ingredients in combinational therapies comprising C/EBPα-saRNA of the present invention and at least one other active ingredient or therapy.

Non-limiting examples of the devices include a pump, a catheter, a needle, a transdermal patch, a pressurized olfactory delivery device, iontophoresis devices, multi-layered microfluidic devices. The devices may be employed to deliver C/EBPα-saRNA of the present invention according to single, multi- or split-dosing regiments. The devices may be employed to deliver C/EBPα-saRNA of the present invention across biological tissue, intradermal, subcutaneously, or intramuscularly. More examples of devices suitable for delivering oligonucleotides are disclosed in International Publication WO 2013/090648 filed Dec. 14, 2012, the contents of which are incorporated herein by reference in their entirety.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

About: As used herein, the term “about” means +/−10% of the recited value.

Administered in combination: As used herein, the term “administered in combination” or “combined administration” means that two or more agents, e.g., saRNA, are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently close together such that a combinatorial (e.g., a synergistic) effect is achieved.

Amino acid: As used herein, the terms “amino acid” and “amino acids” refer to all naturally occurring L-alpha-amino acids. The amino acids are identified by either the one-letter or three-letter designations as follows: aspartic acid (Asp:D), isoleucine threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, the saRNA of the present invention may be considered biologically active if even a portion of the saRNA is biologically active or mimics an activity considered biologically relevant.

Cancer: As used herein, the term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

Cell growth: As used herein, the term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

Cell type: As used herein, the term “cell type” refers to a cell from a given source (e.g., a tissue, organ) or a cell in a given state of differentiation, or a cell associated with a given pathology or genetic makeup.

Chromosome: As used herein, the term “chromosome” refers to an organized structure of DNA and protein found in cells.

Complementary: As used herein, the term “complementary” as it relates to nucleic acids refers to hybridization or base pairing between nucleotides or nucleic acids, such as, for example, between the two strands of a double-stranded DNA molecule or between an oligonucleotide probe and a target are complementary.

Condition: As used herein, the term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a saRNA of the present invention to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides, proteins or polynucleotides, e.g, saRNA, disclosed herein. They may be within the amino acids, the peptides, proteins, or polynucleotides located at the N- or C- termini or 5′ or 3′ termini as the case may be.

Encapsulate: As used herein, the term “encapsulate” means to enclose, surround or encase.

Engineered: As used herein, embodiments of the invention are “engineered” when they are designed to have a feature or property, whether structural or chemical, that varies from a starting point, wild type or native molecule.

Equivalent subject: As used herein, “equivalent subject” may be e.g. a subject of similar age, sex and health such as liver health or cancer stage, or the same subject prior to treatment according to the invention. The equivalent subject is “untreated” in that he does not receive treatment with a saRNA according to the invention. However, he may receive a conventional anti-cancer treatment, provided that the subject who is treated with the saRNA of the invention receives the same or equivalent conventional anti-cancer treatment.

Exosome: As used herein, “exosome” is a vesicle secreted by mammalian cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a saRNA of the present invention and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene: As used herein, the term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

Gene expression: As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Genome: The term “genome” is intended to include the entire DNA complement of an organism, including the nuclear DNA component, chromosomal or extrachromosomal DNA, as well as the cytoplasmic domain (e.g., mitochondrial DNA).

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

The term “hyperproliferative cell” may refer to any cell that is proliferating at a rate that is abnormally high in comparison to the proliferating rate of an equivalent healthy cell (which may be referred to as a “control”). An “equivalent healthy” cell is the normal, healthy counterpart of a cell. Thus, it is a cell of the same type, e.g. from the same organ, which performs the same functions(s) as the comparator cell. For example, proliferation of a hyperproliferative hepatocyte should be assessed by reference to a healthy hepatocyte, whereas proliferation of a hyperproliferative prostate cell should be assessed by reference to a healthy prostate cell.

By an “abnormally high” rate of proliferation, it is meant that the rate of proliferation of the hyperproliferative cells is increased by at least 20, 30, 40%, or at least 45, 50, 55, 60, 65, 70, 75%, or at least 80%, as compared to the proliferative rate of equivalent, healthy (non-hyperproliferative) cells. The “abnormally high” rate of proliferation may also refer to a rate that is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, compared to the proliferative rate of equivalent, healthy cells.

The term “hyperproliferative cell” as used herein does not refer to a cell which naturally proliferates at a higher rate as compared to most cells, but is a healthy cell. Examples of cells that are known to divide constantly throughout life are skin cells, cells of the gastrointestinal tract, blood cells and bone marrow cells. However, when such cells proliferate at a higher rate than their healthy counterparts, then they are hyperproliferative.

Hyperproliferative disorder: As used herein, a “hyperproliferative disorder” may be any disorder which involves hyperproliferative cells as defined above. Examples of hyperproliferative disorders include neoplastic disorders such as cancer, psoriatic arthritis, rheumatoid arthritis, gastric hyperproliferative disorders such as inflammatory bowel disease, skin disorders including psoriasis, Reiter's syndrome, pityriasis rubra pilaris, and hyperproliferative variants of the disorders of keratinization.

The skilled person is fully aware of how to identify a hyperproliferative cell. The presence of hyperproliferative cells within an animal may be identifiable using scans such as X-rays, MRI or CT scans. The hyperproliferative cell may also be identified, or the proliferation of cells may be assayed, through the culturing of a sample in vitro using cell proliferation assays, such as MTT, XTT, MTS or WST-1 assays. Cell proliferation in vitro can also be determined using flow cytometry.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Label: The term “label” refers to a substance or a compound which is incorporated into an object so that the substance, compound or object may be detectable.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form saRNA conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Metastasis: As used herein, the term “metastasis” means the process by which cancer spreads from the place at which it first arose as a primary tumor to distant locations in the body. Metastasis also refers to cancers resulting from the spread of the primary tumor. For example, someone with breast cancer may show metastases in their lymph system, liver, bones or lungs.

Modified: As used herein “modified” refers to a changed state or structure of a molecule of the invention. Molecules may be modified in many ways including chemically, structurally, and functionally. In one embodiment, the saRNA molecules of the present invention are modified by the introduction of non-natural nucleosides and/or nucleotides.

Naturally occurring: As used herein, “naturally occurring” means existing in nature without artificial aid.

Nucleic acid: The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

Patient: As used herein, “patient” refers to a subject who may seek or be in need of treatment, requires treatment, is receiving treatment, will receive treatment, or a subject who is under care by a trained professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemi sulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacologic effect: As used herein, a “pharmacologic effect” is a measurable biologic phenomenon in an organism or system which occurs after the organism or system has been contacted with or exposed to an exogenous agent. Pharmacologic effects may result in therapeutically effective outcomes such as the treatment, improvement of one or more symptoms, diagnosis, prevention, and delay of onset of disease, disorder, condition or infection. Measurement of such biologic phenomena may be quantitative, qualitative or relative to another biologic phenomenon. Quantitative measurements may be statistically significant. Qualitative measurements may be by degree or kind and may be at least 10%, 20%, 30%, 40%, 50%, 60%, 70%, 80%, 90% or more different. They may be observable as present or absent, better or worse, greater or less. Exogenous agents, when referring to pharmacologic effects are those agents which are, in whole or in part, foreign to the organism or system. For example, modifications to a wild type biomolecule, whether structural or chemical, would produce an exogenous agent. Likewise, incorporation or combination of a wild type molecule into or with a compound, molecule or substance not found naturally in the organism or system would also produce an exogenous agent. The saRNA of the present invention, comprises exogenous agents. Examples of pharmacologic effects include, but are not limited to, alteration in cell count such as an increase or decrease in neutrophils, reticulocytes, granulocytes, erythrocytes (red blood cells), megakaryocytes, platelets, monocytes, connective tissue macrophages, epidermal langerhans cells, osteoclasts, dendritic cells, microglial cells, neutrophils, eosinophils, basophils, mast cells, helper T cells, suppressor T cells, cytotoxic T cells, natural killer T cells, B cells, natural killer cells, or reticulocytes. Pharmacologic effects also include alterations in blood chemistry, pH, hemoglobin, hematocrit, changes in levels of enzymes such as, but not limited to, liver enzymes AST and ALT, changes in lipid profiles, electrolytes, metabolic markers, hormones or other marker or profile known to those of skill in the art.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Prognosing: As used herein, the term “prognosing” means a statement or claim that a particular biologic event will, or is very likely to, occur in the future.

Progression: As used herein, the term “progression” or “cancer progression” means the advancement or worsening of or toward a disease or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Protein: A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues are an artificial chemical analogue of a corresponding naturally occurring amino acid.

Protein expression: The term “protein expression” refers to the process by which a nucleic acid sequence undergoes translation such that detectable levels of the amino acid sequence or protein are expressed.

Purified: As used herein, “purify,” “purified,” “purification” means to make substantially pure or clear from unwanted components, material defilement, admixture or imperfection.

Regression: As used herein, the term “regression” or “degree of regression” refers to the reversal, either phenotypically or genotypically, of a cancer progression. Slowing or stopping cancer progression may be considered regression.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

Tumor growth: As used herein, the term “tumor growth” or “tumor metastases growth”, unless otherwise indicated, is used as commonly used in oncology, where the term is principally associated with an increased mass or volume of the tumor or tumor metastases, primarily as a result of tumor cell growth.

Tumor Burden: As used herein, the term “tumor burden” refers to the total Tumor Volume of all tumor nodules with a diameter in excess of 3 mm carried by a subject.

Tumor Volume: As used herein, the term “tumor volume” refers to the size of a tumor. The tumor volume in mm³ is calculated by the formula: volume=(width)²×length/2.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

The invention is further illustrated by the following non-limiting examples.

### EXAMPLES

### Example 1

### Preparations of CEBPA-51 and MTL-CEBPA

Materials and Procedures of preparing CEBPA-saRNAs have been disclosed in WO2015/075557 and WO2016/170349 to MiNA Therapeutics Limited. The preparations of CEBPA-51 and MTL-CEBPA have been disclosed in Examples of WO2016/170349.

In brief, each strand of CEBPA-51 was synthesized on a solid support by coupling phosphoramidite monomers sequentially. The synthesis was performed on an automatic synthesizer such as an Akta Oligopilot 100 (GE Healthcare) and a Technikrom synthesizer (Asahi Kasei Bio) that delivers specified volumes of reagents and solvents to and from the synthesis reactor (column type) packed with solid support. The process began with charging reagents to the designated reservoirs connected to the reactor and packing of the reactor vessel with the appropriate solid support. The flow of reagent and solvents was regulated by a series of computer-controlled valves and pumps with automatic recording of flow rate and pressure. The solid-phase approach enabled efficient separation of reaction products as coupled to the solid phase from reagents in solution phase at each step in the synthesis by washing of the solid support with solvent.

CEBPA-51 was dissolved at ambient temperature in sodium acetate/ sucrose buffer pH 4.0 and lipids were dissolved in absolute ethanol at 55° C. Liposomes were prepared by crossflow ethanol injection technology. Immediately after liposome formation, the suspension was diluted with sodium chloride/phosphate buffer pH 9.0. The collected intermediate product was extruded through polycarbonate membranes with a pore size of 0.2 μm. The target saRNA concentration was achieved by ultrafiltration. Non-encapsulated drug substance and residual ethanol were removed by subsequent diafiltration with sucrose/phosphate buffer pH 7.5. Thereafter, the concentrated liposome suspension was 0.2 μm filtrated and stored at 5±3° C. Finally, the bulk product was formulated, 0.2 μm filtrated and filled in 20 ml vials.

MTL-CEBPA was presented as a concentrate solution for infusion. Each vial contains 50 mg of CEBPA-51 (saRNA) in 20 ml of sucrose/phosphate buffer pH about 7.5.

### Example 2

### CEBPA-51 in Combination with an FGFR4 Inhibitor

1. FGFR4-siRNA
Methods

### Cell Culture

### General

HepB3 cells were grown in DMEM supplemented with 10% fetal bovine serum (FBS), 2 mM L-glutamine, and penicillin/streptomycin in a 5% CO2 incubator. Transfections were performed using 2 μL of Lipofectamine 2000 (Life Technologies) per well in 24 well plate.

### SiFGFR4

The oligos used were as follows: siFGFR4 (s5176, Thermo Fisher Scientific), Negative control oligonucleotide and CEPBA-51. HepB3 cells were seeded at 1×10⁵ cells/well in a 24-well plate. Cells were reverse transfected with oligo at seeding, forward transfected 24 hours later, and RNA collected 72 hours after seeding.

| | |
|---|---|
| siFGFR4 (s5176) | Base sequence: |
| | Sense strand: CAUUGACUACUAUAAGAAATT |
| | (SEQ ID NO: 4) |
| | Anti-sense strand: UUUCUUAUAGUAGUCAAUGTG |
| | (SEQ ID NO: 5) |
| Negative control: | Sense strand: |
| siFLUC | CmUmUAmCGmCmUGAGmUAmCmUmUmCGAdTpsdT |
| | (SEQ ID NO: 6) |
| | Anti-sense strand: UCGAAGmUACUmCAGCGmUAAGdTpsdT. |
| | (SEQ ID NO: 7) |
| | m = 2′-O-Methyl and ps = Phosphorothioate. |
| | All sequences are shown 5′ to 3′. |

### WST-1 Growth Assays

HepB3 cells were seeded at 1×10⁴ cells/well in a 96 well plate, reverse transfected with oligo at seeding and forward transfected 24 hours later. WST cell viability assay was performed as according to the manufactures' instructions (with 24 hr WST incubation). The WST alone signal has been subtracted from the cell-WST signal.

RNA Extraction and qRT-PCR

RNA was isolated from cultured cells using the RNeasy Mini Kit (QIAGEN). RNA was quantitated using a Qiaxpert (Qiagen), and reverse transcribed using the Quantitect Reverse Transcription Kit (QIAGEN). Relative expression levels were determined by qPCR using Quantifast SYBR Green Master Mix (QIAGEN) on a Quantstudio 5 (Qiagen). The following Quantitect Primer Assays (QIAGEN) were used: CEBPA_1_SG, FGFR4_1_SG and GAPDH_1_SG. Relative expression was determined the DDCt method normalized to GAPDH expression.

Results

Effects of single or combination treatment—siFGFR4 and CEBPA-51—on mRNA levels were monitored by qRT-PCR. Treatment of Hep3B cells with a siRNA targeting FGFR4 mRNA reduced FGFR4 mRNA levels significantly. However, treatment with CEBPA-51 had no significant effect on FGFR4 mRNA levels (see Table 6A). Treatment of Hep3B cells with CEPBA-51 or siFGFR4 increased CEPBA mRNA levels. Combination treatment (siFGFR4 and CEPBA-51 in a mixture) further increased CEPBA levels beyond that observed for siFGFR4 or CEPBA-51 alone (see Table 6B). NC: Negative control oligonucleotide.

TABLE 6A
FGFR4-mRNA levels in Hep3B cells

| Transfection | FGFR4-mRNA relative expression |
|---|---|
| Mock | 1.00 |
| siFGFR4 & NC (10 nM each) | 0.10 |
| CEBPA-51 & NC (10 nM each) | 0.91 |

TABLE 6B
CEBPA-mRNA levels in Hep3B cells

| | CEBPA-mRNA |
|---|---|
| Transfection | relative expression |
| Mock | 1.00 |
| CEBPA-51 & NC (10 nM each) | 1.30 |
| siFGFR4 & CEBPA-51 (10 nM each) | 1.75 |
| siFGFR4 & NC (10 nM each) | 1.55 |

Treatment of Hep3B cells with a siRNA targeting siFGFR4 mRNA or CEPBA-51 reduced cell viability. Combination treatment of both siFGFR4 and CEPBA-51 further reduced cell viability as monitored by WST1 growth assay (Table 7A, 96 hours; Table 7B, 120 hours).

TABLE 7A
Normalized cell viability 96 hours after treatment

| | Normalized |
|---|---|
| Transfection | cell viability |
| Mock | 1.00 |
| NC (20 nM) | 0.62 |
| NC & CEBPA-51 (10 nM each) | 0.35 |
| NC & siFGFR4 (10 nM each) | 0.20 |
| CEBPA-51 & siFGFR4 (10 nM each) | 0.14 |

TABLE 7B
Normalized cell viability 120 hours after treatment

| | Normalized |
|---|---|
| Transfection | cell viability |
| Mock | 1.00 |
| NC (20 nM) | 0.70 |
| NC & CEBPA-51 (10 nM each) | 0.20 |
| NC & siFGFR4 (10 nM each) | 0.15 |
| CEBPA-51 & siFGFR4 (10 nM each) | 0.10 |

Conclusion

Combination treatment of siFGFR4 and CEPBA-51 is more effective in increasing CEPBA-mRNA levels in Hep3B cells and consequently reducing cell viability. Overall these data suggest that the combination treatment of FGFR4 inhibitors with CEPBA-51 could be a viable therapeutic strategy for liver cancer.

2. FGFR4-Inhibiting Antibody

In another study, Hep3B cells were seeded at 1.0×10⁵ cells/well in a 24 well plate in the presence of 30 ug/ml anti-human FGFR4 therapeutic antibody (XPA.48.056; Creative Biolabs) as applicable. Cells were transfected with either 10 nM CEBPA-51 saRNA, 10 nM Control oligo (NC) or transfection agent alone (Mock) at seeding and this was repeated 24 hours post- seeding and culture medium changed at 48 hours. FGFR antibody treatment was maintained throughout the experiment. Cells were harvested for RNA collection 72 hours post-seeding qRT-PCR analysis.

As shown in Table 6, the combination of CEBPA-51 and FGFR antibody gave the largest increase in CEBPA mRNA expression.

TABLE 8
CEBPA-mRNA levels in Hep3B cells

| | CEBPA-mRNA |
|---|---|
| Transfection procedure | relative expression |
| Mock, no antibody | 1.00 |
| CEBPA-51 (10 nM), no antibody | 1.31 |
| Mock + antibody | 1.27 |
| CEBPA-51 (10 nM) + antibody | 1.45 |
| NC (10 nM), no antibody | 0.89 |
| NC (10 nM) + antibody | 1.10 |

Treatment of Hep3B cells with FGFR4 antibody reduced cell viability. Combination treatment of both FGFR4 antibody and CEPBA-51 further reduced cell viability as monitored by WST1 growth assay (Table 9A, 96 hours; Table 9B, 120 hours).

TABLE 9A
Normalized cell viability 96 hours after treatment

| | Normalized |
|---|---|
| Transfection | cell viability |
| Mock, no antibody | 1.00 |
| NC (10 nM), no antibody | 0.48 |
| CEBPA-51 (10 nM), no antibody | 0.32 |
| Mock + antibody | 0.69 |
| NC (10 nM) + antibody | 0.34 |
| CEBPA-51 (10 nM) + antibody | 0.22 |

TABLE 9B
Normalized cell viability 120 hours after treatment

| | Normalized |
|---|---|
| Transfection | cell viability |
| Mock, no antibody | 1.00 |
| NC (10 nM), no antibody | 0.59 |
| CEBPA-51 (10 nM), no antibody | 0.25 |
| Mock + antibody | 0.64 |
| NC (10 nM) + antibody | 0.42 |
| CEBPA-51 (10 nM) + antibody | 0.15 |

### Example 3

### CEBPA-51 in Combination with a CEBPB Inhibitor

### CEBPA Activation or CEBPB Suppression Inhibits HCC Cell Migration

Cell migration plays a critical role in cellular processes, including the invasion and metastasis of tumour cells. The effects of CEBPA-saRNA and CEBPB-siRNA on cell migration were investigated to determine whether either of these two treatments would decrease migration in Hep3B cells because the migratory capacity of Hep3B cells is much higher than that of HepG2 cells. A Transwell migration assay was performed to measure migration of the treated cells. The relative cell migrations counted from 9 randomly selected fields under a 10X magnification microscope were recorded. Compared to untransfected Hep3B cells, CEBPA activation or CEBPB repression resulted in significant decreases in cell migration (0.8- or 0.6-fold, respectively).

TABLE 10
Relative Hep3B cell migration

| Treatment | Relative migration |
|---|---|
| Untransfected | 1.00 |
| Scrambled-siRNA | 1.10 |
| Scrambled-saRNA | 1.20 |
| CEBPB-siRNA | 0.25 |
| CEBPA-siRNA | 1.15 |
| CEBPA-saRNA | 0.10 |

### Synergistic Activity of CEBPA-saRNA and CEBPB-siRNA.

To investigate the synergistic activity of CEBPA with its downstream targets CEBPB and p21 in HCC, co-transfections of CEBPA-saRNA with siRNA or saRNA against its downstream targets were performed in HepG2 cells. A transfection of CDKN1A-saRNA was also included, in order to identify if CDKN1A-saRNA would affect p21 activation induced by co-transfections.

Cells in the control groups were untransfected or transfected with 20 nM or 50 nM scrambled saRNA, 10 nM scrambled siRNA+20 nM scrambled saRNA, 10 nM scrambled siRNA+50 nM scrambled saRNA, or 10 nM scrambled siRNA+70 nM scrambled saRNA. Cells with single treatment were transfected with 20 nM CEBPA-saRNA or 50 nM CEKN1A-saRNA. Cells with double combo treatment were transfected with CEBPA-saRNA (20 nM)+CDKN1A-saRNA (50 nM) or CEBPA-saRNA (20 nM)+CEBPB-siRNA (10 nM). Cells with triple combo treatment were transfected with CEBPA-saRNA (20 nM)+CDKN1A-saRNA (50 nM)+CEBPB-siRNA (10 nM).

Compared to untransfected cells, an increase in CEBPA expression (over 2-fold) and a decrease in CEBPB expression (over 0.4-fold) were observed in HepG2 cells co-transfected with CEBPA-saRNA and CEBPB-siRNA. Surprisingly, compared to the other treatments (single, double or triple transfections), the co-transfection of CEBPA-saRNA and CEBPB-siRNA increased the expression of CEBPA, p21 (5.5-fold) and albumin (2.5-fold) the most relative to the untransfected controls. It was estimated that CEBPB inhibition in the presence of CEBPA-saRNA may have a better anti-proliferative response, as it had a greater activation of both CEBPA and p21 than other treatments (single, double or triple transfection) in HCC cells.

The effects of CEBPA-saRNA in combination with CEBPB- siRNA on HCC cell number and proliferation were subsequently investigated using SRB and WST-1 assays in HCC cells. HepG2, Hep3B and PLC/PRFS cells were grown in standard 96-well plates and transfected with 20 nM CEBPA-saRNA, 10 nM CEBPB-siRNA, 20 nM scrambled siRNA, 20 nM scrambled saRNA, scrambled saRNA (10 nM)+scrambled siRNA (10 nM), or scrambled saRNA (20 nM)+scrambled siRNA (10 nM). Cells were also transfected with various combinations of saRNAs and siRNAs to examine potential synergies: CEBPA-saRNA (10 nM)+CEBPB-siRNA (10 nM); or CEBPA-saRNA (20 nM)+CEBPB-siRNA (10 nM). Cytotoxicity was measured using a sulphorhodamine B (SRB) assay. The absolute cell numbers for HepG2 cells, Hep3B and PLF/PRC/5 cells after each treatment were calculated using a titration curve, established on the basis of the OD value measured with a spectrophotometry plate reader. Cell proliferation was assessed using a WST-1 assay and OD values were measured at 10 minute intervals.

20 nM CEBPA-saRNA and 10 nM CEBPB-siRNA both decreased cell number and cell proliferation. 20 nM CEBPA-saRNA worked much better than 10 nM CEBPB-siRNA in HepG2 and Hep3B cells, but only slightly better in PLC/PRF/5 cells. In all cells, the greatest decrease in cell number and cell proliferation was observed after co-transfection with CEBPA-saRNA (20 nM) and CEBPB-siRNA (10 nM). A decrease of cell number after transfection with the combination of CEBPA-saRNA (20 nM)+CEBPB-siRNA (siRNA) was observed, not only in differentiated HepG2 (0.7-fold) and Hep3B (0.8-fold) cells, but surprisingly, also in undifferentiated PLC/PRF/5 (0.65-fold) cells. Additionally, compared to untransfected cells, the combination of CEBPA-saRNA (20 nM) and CEBPB-siRNA (10 nM) decreased relative cell proliferation the most in all cell types. Cell proliferation of undifferentiated PLC/PRF/5 cells was reduced 0.7-fold (70% decrease), similarly to differentiated HepG2 cells (0.8-fold/80% decrease) and Hep3B cells (0.75-fold/75% decrease). These findings indicated that undifferentiated HCC which is not very responsive to CEBPA-saRNA can be reversed to be responsive through increasing the ratio of CEBPA to CEBPB via the functional co-treatment of CEBPA-saRNA and CEBPB-siRNA. CEBPB knockdown may have shifted the balance of differentiated HCC to a highly proliferative phenotype.

### Example 4

### Combination Treatment of MTL-CEBPA Enhances Immunological Anti-Tumor Response of Radiofrequency Ablation and PD-1 Inhibition in a Pre-clinical HCC Model

The PD-1 inhibitor Nivolumab, which causes activation of T-cells and cell-mediated immune responses against tumour cells, gained accelerated FDA approval for second-line treatment of HCC based on a subgroup of the CHECKMATE-040 trial. Patients treated with Nivolumab showed an overall response rate of 14.3% (95% CI: 9.2, 20.8), with 3 complete responses and 19 partial responses. Response duration ranged from 3.2 to 38.2+ months; 91% of responders had responses lasting 6 months or longer and 55% had responses lasting 12 months or longer.

Radiofrequency ablation (RFA) is the process by which tumour is destroyed using heat, generated by a high frequency alternating current and applied through an electrode tip. RFA is one of the standard treatment options for HCC in clinical practice and is associated with a significant survival benefit. Following RFA, the localised coagulation necrosis of the tumour remains in the body and provides proinflammatory signals to induce the release of large amounts of cellular debris that represents a source of tumour antigens which can trigger a host adaptive immune response against the tumour. Evidence suggests that tumour thermal ablation induces modulation of both innate and adaptive immune systems, inducing anti-tumour immune responses through efficient loading of dendritic cells, enhanced antigen presentation and an amplified tumour-specific T-cell response.

This study evaluated whether the oncological efficacy of MTL-CEBPα to HCC may be enhanced by combination treatment with PD-1 inhibition and RFA through synergism in the immuno-modulatory response in a pre-clinical model. The aim of this study was to evaluate the clinical response of combination therapy of MTL-CEBPA, anti-PD-1 & RFA and characterise changes in splenocytes and tumour infiltrating lymphocytes (TILs) following treatment.

### Methods

To investigate any synergistic effect of MTL-CEBPA with RFA and immune checkpoint inhibition, a reverse translation experiment was conducted, where syngeneic BNL hepatocellular carcinoma tumour cells were injected in the two opposite flanks of immunocompetent BALB/c mice (n=8 in each group). Treatments for hepatoma bearing mice included: 1) RFA on one flank (day 0); 2) Immunotherapy (PD-1 inhibition, RMP1-14, BioXCell, West Lebanon, N.H., USA at 200 μg IV/mouse/dose on days 0, 2 & 5); and 3) MTL-CEBPA (3 mg/kg IV/mouse/dose on days 0, 2 & 5) as well as combinations of all 3 interventions.

### Materials and Methods

### Mice

BALB/c mice were purchased from BioLasco Co. (Taipei, Taiwan). Animal studies were performed in compliance with approval from the Institutional Animal Care and Use Committee of College of Medicine, National Taiwan University. Mice were kept in a conventional, specific pathogen-free facility.

### Tumor Cell Line

BALB/c-derived murine hepatocellular carcinoma cell line BNL 1ME A.7R.1 (BNL; ATCC, Manassas, Va., USA) was cultured in DMEM supplemented with 10% fetal bovine serum (FBS) and antibiotics (penicillin 100 units/mL, streptomycin 100 μg/mL and amphotericin 25 μg/mL) (Gibco BRL, USA). Cells were grown at 37° C. in a 5% CO₂ humidified incubator.

### Animal Models

64 male BABL/c mice (6-weeks-old; from BioLasco Co., Taiwan) were xenografted in bilateral flanks by subcutaneous (s.c.) injection of 50 μL of BNL cell suspension containing 5×10⁵ cells.

### Experimental Groups

Mice were randomly allocated to one of the following 8 experimental groups (8 animals/group):
1. Control group
2. RFA group (R): treated with RFA only
3. Anti-I′D I group (P): treated with anti-PD1 only
4. CEBPα group (C): treated with CEBPa only
5. Anti-PD1+CEBPa group (P+C): treated with CEBPα and anti-PD1
6. RFA+anti-PD1 group (R+P): treated with RFA and anti-PD1
7. RFA+CEBPα (R+C) group: treated with RFA and CEBPa
8. RFA+Anti-PD1+CEBPa group (R+P+C): treated with RFA, CEBPa and anti-PD1

### Treatment Schedule

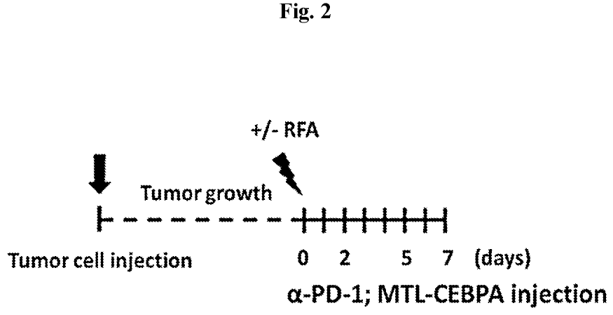

Four weeks after cancer-cell injection, when the tumour reached the diameter of ˜1.5×1.5 cm, one of the bilateral tumours was treated by RFA as day 0. MTL-CEBPA (3 mg/kg) was given by intravenous (i.v.) injection on days 0, 2 & 5 post RFA treatment. Anti-PD-1 (RMP1-14, BioXCell, West Lebanon, N.H., USA), at 200 μg/mouse/dose, was given intraperitoneally (i.p.) on days 0, 2 & 5 post RFA treatment. The tumor sizes were assessed using microcalipers, and the tumor volumes were calculated using the following equation: volume=length×(width)²×0.5. Mice were sacrificed on day 7. Timeline of study design is shown in FIG. 2 (with animals sacrificed on day 7).

### Radiofrequency Ablation (RFA) Treatment

Animals were anaesthetised with intraperitoneal injection of Ketamine/Xylazine solution and positioned prone. After shaving the area, a 22-gauge needle with a 4-mm active tip electrode and EUS-radiofrequency (RF) ablation system (Rita) was used for energy delivery by inserting it into the right flank tumour. A 500-kHz RF generator was used to maintain an output of 10W. Treatment varied from 1 to 3 minutes depending on the tumour volume. Seven days after RFA, mice were scarified with collection of the left frank tumor and spleen to prepare tumor-infiltrating lymphocytes and splenocytes for further analysis. Peripheral blood samples were obtained in heparin-containing tubes before and after treatment.

### Splenocytes and Tumor Infiltrating Lymphocytes Isolation

To isolate murine splenocytes, spleen was extracted and pressed through nylon cell strainer of 40-μm mesh and red cells were lysed with RBC lysis buffer (eBioscience, San Diego, Calif.). To prepare tumor infiltrating lymphocytes (TILs), tumors were harvested and dissected into approximately 5-mm fragment followed by agitation in 0.05 mg/ml collagenase IV and 0.01 mg/ml DNase I in RPMI medium at 37° C. for 40 minutes. Tumors were minced and filtered through 70-μm and 40-μm nylon mesh to remove debris. Cells were then separated on a Ficoll-Hypaque gradient and used for further analysis.

### Flow Ctometry

Spleen and tumor tissues were processed, brought to single cell suspensions in PBS with 0.5% BSA and stained at 4° C. for 30 minutes. The cell surface markers were stained with fluorescent-labeled antibodies: FITC-CD45, PE-CD8, PerCP-CD3, CD49b, and APC.Cy7-CD4 from BD Biosciences (San Jose, Calif.). Cells were then washed twice and fixed with a buffer (BD Biosciences, San Jose, Calif.). The total numbers of individual leukocyte subsets were determined using 123 count eBeads counting beads (eBioscience, San Diego Calif.). Flow cytometry was performed by FACSVerse™ (Becton Dickinson, Mountain View, Calif.) and the data were processed using FlowJo™ software (Ashland, Oreg.).

### Statistics Analysis

Data were presented as means±SD. Statistical significance was assessed by the two-tailed Student's t-test. The differences were considered significant when the p value was less than 0.05. GraphPad Prism (GraphPad Software Inc., San Diego, Calif., USA) was used for the analyses.

### Results

As can be seen in Table 11, combination treatment with RFA appears to improve the therapeutic response to all treatments and their combinations and the best response was seen in group 8 with 2/8 animals showing a complete and 5/8 animals a partial response.

TABLE 11
Therapeutic response to contralateral flank tumour.

| Group | CR | PR | | SD | | PD | |
|---|---|---|---|---|---|---|---|
| 1. Control | 0 | 0 | | 0 | | 8 | (100%) |
| 2. R | 0 | 0 | | 0 | | 8 | (100%) |
| 3. P | 0 | 0 | | 3 | (33.3%) | 6 | (66.7%) |
| 4. C | 0 | 3 | (33.3%) | 3 | (33.3%) | 3 | (33.3%) |
| 5. P + C | 0 | 2 | (22.2%) | 4 | (44.4%) | 3 | (33.3%) |
| 6. R + P | 0 | 0 | | 5 | (50%) | 5 | (50%) |
| 7. R + C | 0 | 4 | (50%) | 2 | (25%) | 2 | (25%) |

| | | | | | | |
|---|---|---|---|---|---|---|
| 8. R + P + C | 2 (25%) | 5 | (62.5%) | 1 | (2.5%) | 0 |

CR—complete response,
PR—partial response,
SD—stable disease,
PD—progressive disease

All animals completed their designated treatment allocation. Mice treated with MTL-CEBPA retarded the growth of tumours compared with untreated control (p<0.01), however; anti-PD-1 alone had a small non significant effect on tumour growth. In contrast the combination of CEBPa and anti-PD-1 did result in a significant antitumour effect compared to the control group which was further enhnaced by the addition of RFA (Table 12).

TABLE 12
Mean change in tumour volume in the experimental groups

| | Median volume change (%) | p vs. control |
|---|---|---|
| Control (C) | 260.4 ± 45.4 | — |
| MTL-CEBPA (M) | 150.5 ± 29.4 | 0.001 |
| anti-PD1 (P) | 219.1 ± 57.1 | n.s |
| anti-PD1 + MTL-CEBPA (P + M) | 169.8 ± 31.6 | 0.0005 |
| RFA (R) | 273.1 ± 63.8 | n.s |
| RFA+ MTL-CEBPA (R + M) | 193.2 ± 54.2 | 0.029 |
| RFA + anti-PD1 (R + P) | 209.0 ± 61.1 | n.s |
| RFA + MTL-CEBPA + | 118.6 ± 33.6 | 0.0006 |
| anti-PD1 (R + P + M) | | |

RFA treatments successfully ablated all the assigned flank tumours, however in the RFA alone treatment group it was seen that the growth of contralateral non-RFA-treated tumours were not significantly affected. For the treatment groups with RFA, the tumour response of CEBPA was significantly better than anti-PD-1. The combined MTL-CEBPA with anti-PD-1 treatment group resulted in 7/8 tour responses (CR and PR) including 2 CRs and had by far the best treatment response related to tumour volume. This triple combination also showed a modest decrease rate of tumour growth relative to MTL-CEBPA alone.

### MTL-CEBPA Enhances CD8+ and NKT Cells Infiltrating in Tumour

To further explore whether the immune response contributes to the potential anti-tumour effect, splenocytes and tumour infiltrating lymphocytes (TILs) from mice after RFA and/or drug treatment groups were measured by flow cytometry. As can be seen from Table 3 MTL-CEBPA combined with anti-PD-1 treatment induced a significant increase of CD4+ and CD8+ lymphocytes in splenocytes but not in TILs. After induction of RFA, the CD8+ tumour infiltration increased significantly in animals treated with triple combination (Group 8).

TABLE 13
Proportion of tumour infiltrating lymphocytes in treatment groups

| | Mean +/− SD of Tumour Infiltrating Cells (% of cells /CD45+) | | | |
|---|---|---|---|---|
| | CD4 | CD8 | NK | NKT |
| C | 93.4 ± 1.59 | 24.8 ± 5.9 | 7.83 ± 3.23 | 5.01 ± 1.30 |
| M | 104.1 ± 5.42 | 34.9 ± 2.9 | 10.9 ± 1.98 | 3.92 ± 0.56 |
| P | 93.4 ± 1.59 | 28.5 ± 2.18 | 9.33 ± 1.82 | 3.95 ± 0.45 |
| P + M | 89.5 ± 6.03 | 45.2 ± 3.45 | 17.6 ± 1.50* | 5.36 ± 0.23 |
| R | 128.3 ± 21.2 | 45.8 ± 6.57 | 13.5 ± 2.93 | 5.16 ± 0.50 |
| R + M | 150.2 ± 20.9 | 67.2 ± 7.19 | 16.0 ± 2.34 | 6.09 ± 0.13 |
| R + P | 95.3 ± 12.3 | 26.9 ± 1.96 | 12.7 ± 0.02 | 5.18 ± 0.20 |
| R + P + M | 116.2 ± 5.9 | 98.9 ± 2.23 | 21.2 ± 2.58* | 8.23 ± 0.32*** |

*P < 0.05,
***p < 0.005

TABLE 14
Proportion of splenocytes in treatment groups

| | Mean +/− SD of Splenocytes (% of cells /Lym) | | | | |
|---|---|---|---|---|---|
| | Lymphocyte | CD4 | CD8 | NK | NKT |
| C | 23.9 ± 3.30 | 5.74 ± 1.72 | 2.4 ± 0.6 | 1.9 ± 0.32 | 0.6 ± 0.24 |
| M | 30.8 ± 1.54* | 11.0 ± 1.60** | 6.3 ± 1.28*** | 2.7 ± 1.50 | 0.6 ± 0.09 |
| P | 26.6 ± 2.49 | 8.68 ± 2.31* | 3.3 ± 0.92 | 2.4 ± 0.85 | 0.6 ± 0.20 |
| P + M | 29.7 ± 3.02 | 9.81 ± 1.91 | 2.6 ± 1.21 | 1.6 ± 0.69 | 0.4 ± 0.05 |
| R | 23.5 ± 1.00 | 6.71 ± 0.65 | 4.5 ± 0.98 | 2.1 ± 0.67 | 0.5 ± 0.17 |
| R + M | 25.4 ± 5.38 | 8.56 ± 3.46 | 5.6 ± 1.01* | 1.9 ± 0.56 | 0.4 ± 0.08 |
| R + P | 23.2 ± 2.87 | 8.93 ± 3.18 | 4.3 ± 2.00 | 2.9 ± 1.26 | 0.5 ± 0.26 |
| R + P + M | 29.6 ± 3.09 | 10.8 ± 1.55* | 5.7 ± 1.78* | 2.5 ± 0.54 | 0.3 ± 0.07 |

*P < 0.05,
***p < 0.005

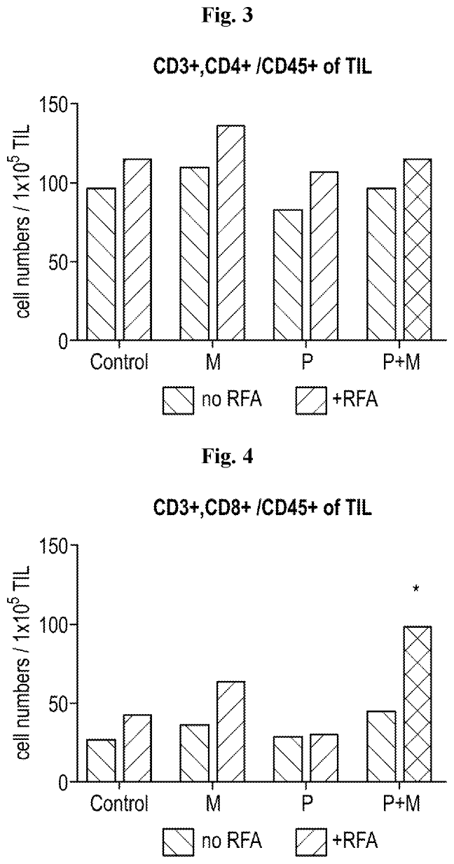

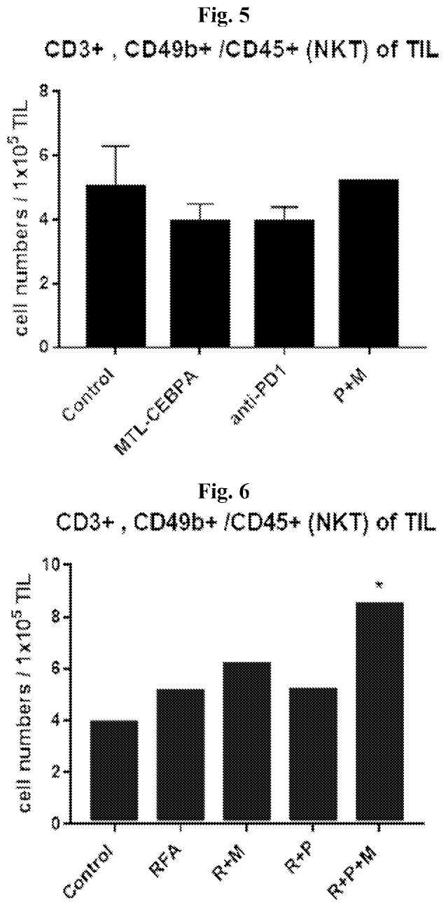

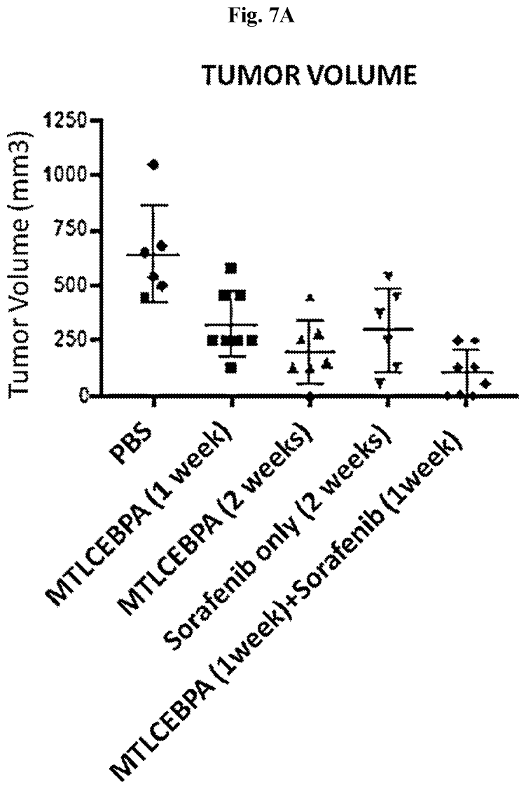

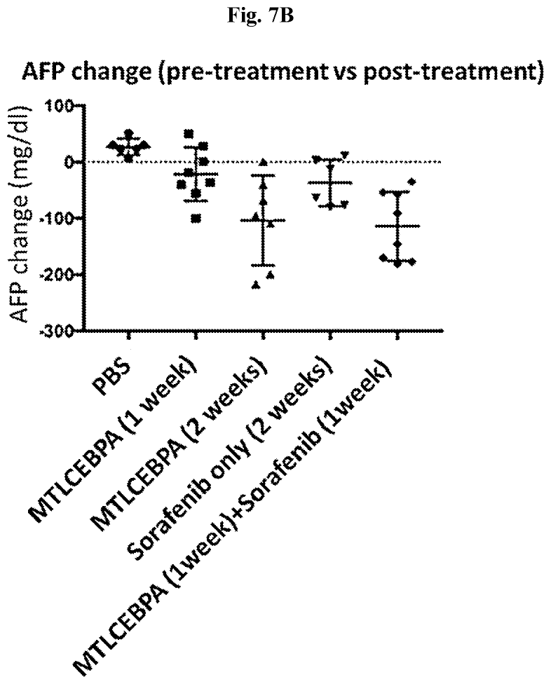

Changes in tumour infiltrating helper T lymphocytes are shown in FIG. 3. Changes in tumour infiltrating cytotoxic T lymphocytes are shown in FIG. 4. Changes in tumour infiltrating Natural Killer T cells without RFA treatment are shown in FIG. 5. Changes in tumour infiltrating Natural Killer T cells with RFA treatment are shown in FIG. 6.

NK and NKT lymphocytes count in spleen and tumour was not significantly different among the treatment groups without RFA, however, an increasing of NKT lymphocytes in TILs and splenocyte in the MTL-CEBPA, anti-PD-1 with RFA combination treatment group was observed.

### Discussion

From mechanistic evaluation of HCC patients treated with MTL-CEBPA, it was observed that it induces a marked reversible and repeatable increase in peripheral granulocytes. qPCR analysis of these cells showed increased mRNA levels of CEBPA and down regulation of PD-L1, adenosine deaminase and CXCR4 suggesting the potential for an immune-modulatory effect on the Tumour Immune Micro Environment (TIME). This observation led to the hypothesis that the clinical efficacy may be further augmented by therapies that synergistically influence the TIME to produce an enhanced immune response to the tumour.

MTL-CEBPA treatment reduced the growth of mouse HCC flank tumours compared with control both with and without combination treatment with RFA. However, the greatest therapeutic response was seen in the group treated with a combination of MTL-CEBPA, PD-1 inhibitor and RFA. This was also the only group in which a proportion of the animals exhibited a complete response to treatment. As the tumour evaluation in animals treated with RFA were all on the contralateral side this suggests that RFA treatment resulted in an abscopal effect, i.e., regression of distant tumour sites owing to induction of T cell responses. PD-1 inhibition on its own or in combination with RFA did not significantly decrease the tumour growth compared to control in this study.

In this study, a significant increase in the tumour associated cytotoxic and natural killer T lymphocytes in the contralateral tumour following treatment with RFA in combination with CEBPA and PD-1 inhibition was observed. A significant therapeutic response to PD-1 inhibition was not detected in this study. However, there was a clear incremental benefit when this was combined with CEBPa and RFA.

In summary, it was observed that combination treatment of MTL-CEBPA enhances the immunological anti-tumour response of radiofrequency ablation and PD-1 inhibition in a pre-clinical HCC model. These data suggest a clinical role for combination treatment with checkpoint blockade, RFA and MTL-CEBPA through synergistic priming of the immune tumour response, enabling RFA to have an abscopal effect.

### Example 5

### MTL-CEBPA Combined with Sorafenib in Treating Advanced Liver Cancer

Sorafenib (Nexavar®), a multikinase inhibitor which targets Raf kinases as well as VEGFR-2/-3, PDGFR-beta, Flt-3 (FMS-like tyrosine kinase-3) and c-Kit, received FDA and EMEA approval for treatment of patients with advanced hepatocellular carcinoma (HCC). However, the low tumour response rates and the side effects associated with this monotherapy indicates the need to investigate other new therapeutic options.

### Materials and Methods

### Animal Models and Treatments

Male Wistar rats (150-180 g) at 7 weeks of age were obtained from the Animal Center of National Taiwan University. The rats were housed in standard conditions, and all the experiments were conducted in accordance with the “Guide for the Care and Use of Laboratory Animals” prepared by the Institutional Animal Care and Use Committee of National Taiwan University. The rats were given DEN solution daily (Sigma, St Louis, Mo.) as the sole source of drinking water for 6 weeks, followed by 3 weeks of regular water. The DEN solution feeding started with 100 ppm in the first week. The average bodyweight (BW) of the animals was measured once a week per group of five rats, and the concentration of DEN in their drinking water was adjusted in proportion to the BW each week relative to that of the first week. For example, if the average BW values at weeks 1, 2 and 3 of DEN administration were 150, 200 (1.3-fold), and 250 g (1.66-fold), respectively, then the DEN concentration in the drinking water was set at 100, 133, and 166 ppm, respectively. After 6 weeks of DEN administration, the animals were given regular water for another 3 weeks and observed, so as to allow sufficient time for tumor progression. The rats were randomly separated in 5 groups of 10 animals/group:
1. Ctrl group: treated with PBS only, i.v. 3×/week for 2 weeks (day1,3,5 and day 8,10,12)
2. MTL-CEBPA group: treated by MTL-CEBPA i.v. 3×/week for 1 week (day1,3,5)
3. MTL-CEBPAX2 group: treated by MTL-CEBPA i.v. 3×/week for 2 weeks (dayl,3,5 and day 8,10,12)
4. Sorafenib group: treated by sorafenib P.O. 10 mg/kg (Nexavar, Bayer), 3×/week for 2 weeks (day1,3,5 and day 8,10,12)
5. MTL-CEBPA+Sorafenib group: treated by MTL-CEBPA i.v. 3×/week for 1 weeks (day1,3,5) and Sorafenib P.O. 10 mg/kg, 3×/week for 1 weeks (day8,10,12)

On 15^{th} day after treatment, the animals were sacrificed and tumour size and tumour/liver weight were measured. The body, liver, lung, and spleen were weighed, and the aspects of all organs were recorded. After the animals were sacrificed, all liver lobes were promptly removed and weighed, and the diameters of all of the macroscopically visible nodules on the liver surface and in the 5-mm sliced sections were measured. Tumour burden was determined in terms of two criteria: the ratio of liver weight/BW, and the total volume of all the tumour nodules with diameter >3 mm.

### Serum Profiles

The serum levels of ALT, AST and total bilirubin were measured with VITROS 5.1 FS Chemistry Systems (Ortho-Clinical Diagnostics, Inc.). The serum level of rat alpha-fetoprotein was evaluated with anti-Rat AFP ELISA Kit (USCN life company/China), following the instructions of the manufacturer.

### Statistics Analysis

Data were presented as means±SD. Statistical significance was assessed by the two-tailed Student's t-test. The differences were considered significant when the p value was less than 0.05. GraphPad Prism (GraphPad Software Inc., San Diego, Calif., USA) was used for these analyses.

### Results

All animals were terminated at the end of the study for liver and blood serum analysis. Liver lobes were removed and weighed, where the diameters of all of the macroscopically visible nodules on the liver surface and in the 5-mm sliced sections were measured. Tumour volume was determined following two criteria: the ratio of liver weight/BW, and the total volume of all the tumour nodules with diameter >3 mm.

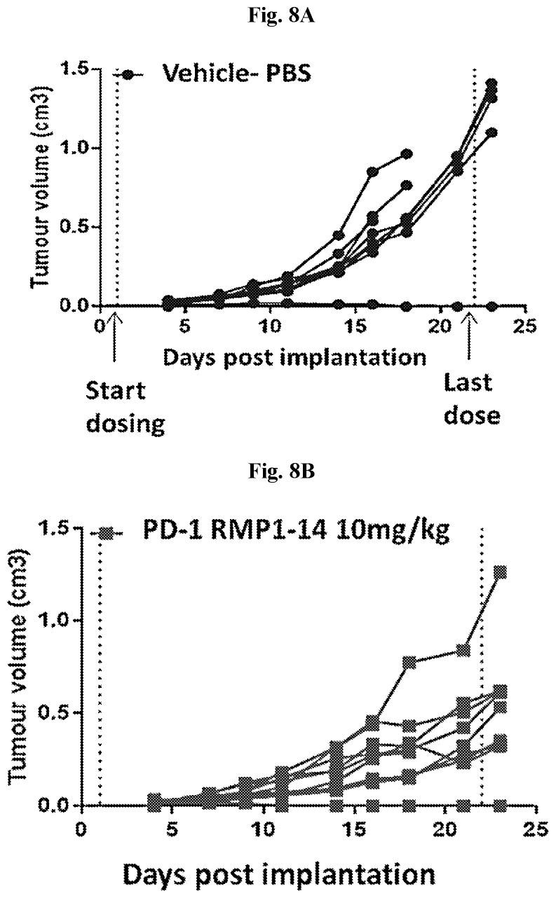

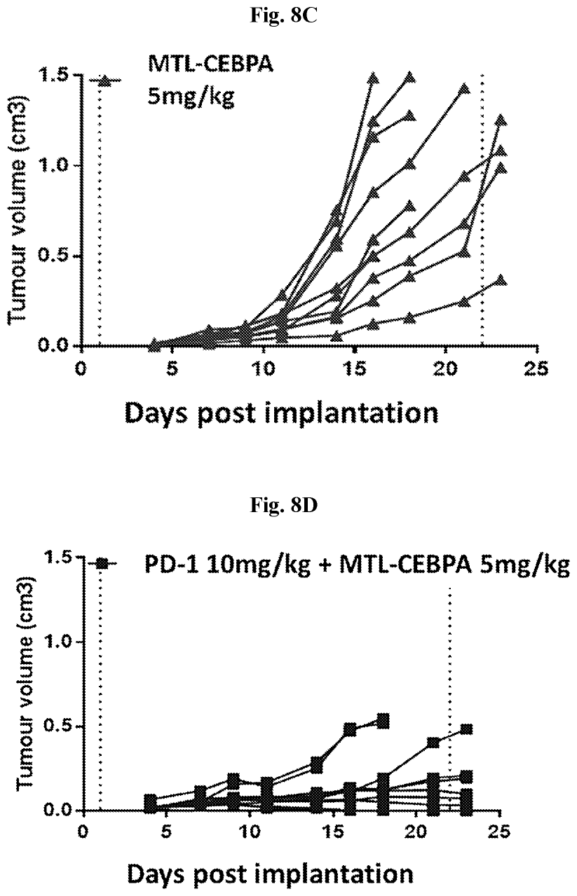

The tumour size in the PBS control group averaged at 644.7 mm³, whilst the tumour size in animals treated with MTL-CEBPA after one week averaged at 326 mm³. Animals treated with MTLCEBPA for two weeks had tumour size averaging at 199.7 mm³. Animals treated with sorafenib for two weeks had tumour size averaging at 299.5 mm³ whilst animals treated with MTL-CEBPA and Sorafenib had tumour size averaging at 101.3 mm³ (FIG. 7A) and Table 15.

TABLE 15
Tumor volume

| | | MTL-CEBPA | MTL-CEBPA | Sorafenib only | MTL-CEBPA (1 week) + |
|---|---|---|---|---|---|
| | PBS | (1 week) | (2 weeks) | (2 weeks) | Sorafenib (1 week) |
| Number of | 6 | 8 | 7 | 6 | 8 |
| Animals | | | | | |
| Mean | 644.7 | 326.0 | 199.7 | 299.5 | 101.3 |
| Std. Deviation | 216.9 | 150.0 | 144.8 | 188.9 | 106.0 |
| Std. Error of | 88.6 | 53.0 | 54.7 | 77.1 | 37.5 |
| Mean | | | | | |
| Unpaired test with | | | | | |
| Welch's correction | | | | | |
| (relative to PBS) | | | | | |
| P value | | 0.014 | 0.002 | 0.015 | 0.001 |
| P value | | * | ** | * | *** |
| summary | | | | | |
| Significantly | | Yes | Yes | Yes | Yes |
| different | | | | | |
| (p < 0.05)? | | | | | |
| One-or two- | | Two | Two | Two | Two |
| tailed P value? | | | | | |

Serum levels of alpha-fetoprotein (AFP) were measured before treatment and compared to measurement after treatment and represented as ‘AFP change’ (mg/dl). Serum AFP change was measured in each group. Values represent measurements before treatment subtracted with after treatment. Significant AFP changes across all treated animals were observed, where values significantly decreased after MTL-CEBPA treatment, sorafenib treatment or the combination of both (FIG. 7B). The most dramatic reduction observed were from animals treated with MTL-CEBPA for 2 weeks or with a combination of MTL-CEBPA and sorafenib (FIG. 7B) and Table 16.

TABLE 16
AFP change (pre-treatment vs post-treatment)

| | | MTL-CEBPA | MTL-CEBPA | Sorafenib | MTL-CEBPA (1 |
|---|---|---|---|---|---|
| | | (1 week | (2 week | only | week) + Sorafenib |
| | PBS | dose) | dose) | (2 weeks) | (1 week) |
| Number of | 6 | 8 | 7 | 6 | 8 |
| Animals | | | | | |
| Mean | 27 | −21.5 | −103.9 | −36.83 | −114 |
| Std. Deviation | 15 | 47.78 | 79.91 | 41.82 | 61.09 |
| Std. Error of | 6 | 16.89 | 30.2 | 17.07 | 21.6 |
| Mean | | | | | |
| Unpaired test with | | | | | |
| Welch's correction | | | | | |
| (relative to PBS) | | | | | |
| P value | | 0.00245 | 0.0045 | 0.0054 | 0.0002 |
| P value | | * | ** | ** | *** |
| summary | | | | | |
| Significantly | | Yes | Yes | Yes | Yes |
| different | | | | | |
| (p < 0.05)? | | | | | |
| One-or two- | | Two | Two | Two | Two |
| tailed P value? | | | | | |

Serum alanine aminotransferase (ALT) and aspartate aminotransferase (AST) and total bilirubin did not deteriorate over the course of the 2-week treatment suggesting that combination treatment does not mediate hepatotoxic effects in the animals and may even enhance the benefits of reducing tumour burden in vivo.

### Discussion

HCC is the second commonest of cancer death in the world with an estimated overall 5 year survival of 12% for all stages of the disease. A small number of patients with limited disease and a background of cirrhosis are suitable for liver transplantation. Curative liver resection is only feasible in less than 20% of patients and systemic therapy is usually reserved for patients not suitable or who have progressed on local treatments such as radiofrequency ablation or transarterial chemo-embolisation.

For a decade sorafenib was the only systemic therapy for advanced HCC as it induces a 31% decrease in the risk of death with a median survival of 10.7 months vs 7.9 months for placebo in patients with advanced disease. During that decade no other systemic drug was approved until some two years ago when several drugs for HCC were approved by the FDA including Levantinib, regorafenib. In September 2017 Nivolumab from Bristol-Meyers Squibb received accelerated approval by the FDA in patients previously treated with sorafenib. This was based on a 154 patient subgroup of the CHECKMATE-040 trial which showed an overall response rate of 14.3% (95% CI: 9.2, 20.8), with 3 complete responses and 19 partial responses. Response duration ranged from 3.2 to 38.2+ months; 91% of responders had responses lasting 6 months or longer and 55% had responses lasting 12 months or longer.

Over the years several researchers tried to improve the efficacy of sorafenib by combining it with other agents. In this study, it is shown that MTL-CEBPA can improve the efficacy of sorafenib in the HCC rat DEN model.

### Example 6

### CEBPA-51 Combo Therapies in Treating Advanced HCC Patients

In an initial clinical study of MTL-CEBPA, a total of 20 advanced HCC patients were stratified into 3 groups. The first group (Group I) had 3 patients, who had received FGFR4 inhibitor (U3-1784, BLU-554) before MTL-CEBPA was administered. The second group (Group II) had 9 patients, who had received ICB (Pembroluzimab ,Tremelimumab, Durvalumab or Nivolumab) before MTL-CEBPA was administered. The third group (Group III) had 8 patients, who had received TKI therapy (7/8 sorafenib and 1/8 sorafenib plus lenvatinib) before MTL-CEBPA was administered.

One patient in Group I showed partial response (PR). Two other patients in Group I showed prolonged stable disease (SD) greater or equal to 6 months. In Group II, 7 patients showed SD and 5 of these patients had an SD for greater or equal to 4 months while only 2 showed progressive disease (PD) at the 2-month MRI scan. In Group III, no patients had SD for greater than 2 months, 4 patients showed SD for 2 months and 4 patients had had PD at the 2 month-MM scan. All the responses are categorized according to Response Evaluation Criteria In Solid Tumors (RECIST). PR=at least a 30% decrease in tumor lesions; SD=neither sufficient shrinkage to qualify for PR nor sufficient increase to qualify for PD; PD=at least 20% increase in tumor lesions.

Hence, pretreatment with an FGFR4 inhibitor or an ICB agent showed benefit compared with standard of care TKI treatment only.

In another clinical study, three patients received tyrosine kinase inhibitors, subsequent to treatment with MTL-CEBPA. Two patients administered with sorafenib experienced confirmed complete tumour responses together with marked decreases in alpha-fetoprotein tumour marker. The first patient has HCC and HepC with cirrhosis. Prior to the study, he received multiple transarterial chemoembolization (TACE) treatments in 2014 to 2017, durvalumab from June 2017 to August 2017 and had disease progression. He received MTL-CEBPA treatment at 98 mg/m² QW×6 from September 2017 to November 2017; transarterial embolization (TAE) in November 2017, and sorafenib from January 2018 to May 2018. He had partial response in March 2018, complete response in May 2018, and complete response in July 2018. He experienced resolution of HCC and both lung and peritoneal metastasis. The second patient has HCC and HepB with cirrhosis. Prior to the study, he received ablation in 2014-2017, TACE/doxorubicin (refractory) in 2016-2017. He received MTL-CEBPA at 130 mg/m² QW×6 from November 2017 to January 2018, TACE in April 2018, and sorafenib since April 2018 (ongoing). He had complete response as of May 2018 and August 2018. A further 3 patients who also received sorafenib subsequent to MTL-CEBPA. One had a complete response (liver and lung metastases), one had a Partial Response and one had progressive disease. The interval between MTL-CEBPA and Sorafanib treatment where complete or partial responses were seen ranged from 0-3 months.

One patient administered with lenvatinib subsequent to treatment with MTL-CEBPA experienced a partial tumour response.

In previously published Phase III studies as single agents, complete responses were observed in 0% of patients treated with sorafenib and partial responses were observed in 2% of patients treated with sorafenib and 24% of patients treated with lenvatinib. The 3 out of 5 complete responses (60%) and 4 out of 5 complete and partial responses (80%) with post treatment sorafenib compared to historical data of 0% and 2% respectively with sorafenib alone shows that MTL-CEBPA has great potential to enhance the benefits of other cancer therapies including TKIs.

### Example 7

### A Study to Assess the Activity of MTL-CEBPA in Combination with a PD-1 Antibody in a Syngeneic CT26 Colorectal Cancer Model

In this study, whether MTL-CEBPA could enhance the activity of a PD-1 checkpoint antibody in a commonly used mouse syngeneic model CT26 was investigated.

### Brief Methods:

Female Balb/c (6 weeks old) were transplanted subcutaneously (s.c.) with 0.1 ml of the murine colorectal cancer cell line CT26.WT (CRL-2638) at 5×10⁵ cells per mouse. CT26.WT cells were grown in RPMI-1640 media with 10% FCS and 2 mM glutamine at 37° C. and 5% CO₂. The order of the cell implant was randomised by box. Dosing commenced on day 1 post cell implant and animals were given 7 doses in total of:
1). MTL-CEBPA alone (5 mg/kg i.v.) given on a d1 and d3 schedule;
2). PD-1 RMP1-14 antibody alone (10 mg/kg i.p.) given on a d1 and d4 schedule;
3). MTL-CEBPA combined with PD-1 antibody with same dose and schedule as above; or
4). PBS alone on same dose and schedule as the combination therapy group.

Tumours were measured three times weekly by calliper and volume of tumours calculated using elliptical formula (pi/6×width×width×length). At the end of study flash frozen tumour samples (and FFPE fixed sample if sufficient tumour) were taken 24 hrs post last dose. A serum sample was also taken and flash frozen. Where possible if animals were terminated early tumour samples and serum were also obtained for analysis.

For Nanostring analysis RNA was isolated from the flash frozen samples. Tissue samples were homogenised in QIAzol lysis reagent (Qiagen), 1-Bromo-3-chloropropane (Sigma) was added to each sample and vortexed followed by centrifugation in a pre-chilled centrifuge at +4° C. The aqueous upper phase was then transferred into an Ultra recovery tube (Starlab I1420-2600) containing ethanol and the samples were mixed by gentle pipetting. The samples were then transferred into RNeasy columns (Qiagen 74106) and RNA extraction was performed according to the instructions in the kit and RNA was quantified using the QIAxpert system.

Nanostring analysis was carried out using a Nanostring machine and using Nanostring Mouse 360 IO codeset-LBL-10545-01 and mouse myeloid innate Immunity codeset- LBL-10398-02 chips.

### Main Results of the Studies:

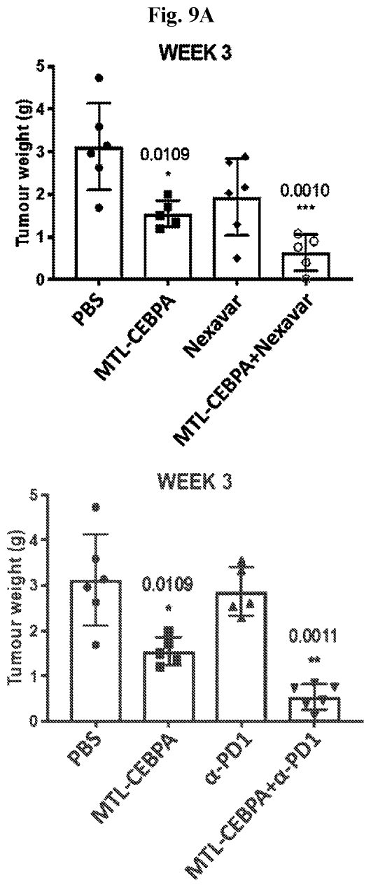

The individual tumour plots and scatter plots at day 18, 21 and 23 for the PBS, PD-1 alone, MTL-CEBPA alone and PD1 plus MTL-CEBPA are shown in FIG. 8A, 8B, 8C and 8D. The size of the tumours in the MTL-CEBPA alone group were not significantly different from the PBS group at day 18, day 21 or day 23. In the PD1 antibody alone group the mean tumour size was significantly less than PBS at day 18 only. In contrast at day 18, 21 and 23 the MTL-CEBPA and PD-1 antibody combo group were significantly (P<0.05—unpaired t-test with Welch's correction) smaller than the PBS. At day 21 and day 23 the MTL-CEBPA plus PD-1 antibody combo group tumours were significantly (P<0.05) smaller than either the PD1 antibody or MTL-CEBPA alone group demonstrating the anti-tumour enhancement of the combination. In the MTL-CEBPA plus PD-1 antibody combination group at day 23 only 3/6 of the remaining tumours were starting to increase in size from the day 21 measurement whereas remaining tumours in both the MTL-CEBPA and PD-1 antibody alone groups were all increasing in size from day 21 to day 23.

Nanostring analysis of tumours at day 23 using CEBPA probe on the Myeloid chip revealed approximately a 1.7 fold (P<0.001 vs PBS group) increase in CEBPA mRNA in the MTL-CEBPA alone group. In contrast the level of CEBPA mRNA in tumours treated with PD-1 antibody was unchanged (1.09; P=0.705 vs the PBS group). In the MTL-CEBPA plus PD-1 antibody combination group overall the mean increase in CEBPA mRNA was 5.12 fold; in the 3 tumours that were starting to grow the mean increase in level of CEBPA mRNA was 1.56(P=0.283 vs PBS group) and in the 3 tumours that were still decreasing in size the level of CEBPA mRNA was 8.69(P<0.001 vs the PBS group) (Table 17). In terms of CD8 T-cell marker CD8a (IO chip) only the combination group showed a significant increase overall (3.7 fold, P<0.02) and the increase was most marked in the 3 regressing tumours (5.05 fold, P<0.02 vs PBS). There was a small increase in CD8a mRNA in the PD1 antibody alone group (1.29 fold) and the MTL-CEBPA group (1.22 fold) that weren't statistically significant vs the PBS control group (Table 17). Looking at Granzyme A mRNA (myeloid chip) levels indicative of activated CD8+ve, there was a small increase in the PD-1 antibody alone group (1.38 fold) and MTL-CEBPA alone group (1.14 fold), neither being significant while overall in the combination group there was a 2.39 fold increase (P=0.05 vs PBS) and this was most marked in the 3 regressing tumours (3.22 fold−p<0.05).

TABLE 17
Increases in CEBPA mRNA and CD8a mRNA measured by nanostring

| Treatment | CEBPA mRNA | CD8a mRNA | Granzyme A mRNA |
|---|---|---|---|
| (number of tumours | Fold increase and p-value | Fold increase and p-value | Fold increase and p- |
| analysed) | vs PBS | vs PBS | value vs PBS |
| MTL-CEBPA (6) | 1.74 − p < 0.002 | 1.22 − p = 0.20 | 1.14 − p = 0.62 |
| PD1 antibody (4) | 1.09 − p = 0.71 | 1.29 − p = 0.12 | 1.38 − p = 0.24 |
| MTL-CEBPA plus PD1 | 8.69 − p < 0.001 | 5.05 − p < 0.02 | 3.22 − p < 0.05 |
| antibody regressing (3) | | | |
| MTL-CEBPA plus PD1 | 1.56 − p = 0.283 | 2.31 − p = 0.10 | 1.55 − p = 0.134 |
| antibody progressing (3) | | | |

### Conclusions

Overall the data indicate a benefit in terms of anti-tumour activity of combining MTL-CEBPA with PD-1 antibody in this immunocompetent mouse CT26 colorectal cancer model. Activity was accompanied by an increased expression of CEBPA mRNA (likely in stromal cells since CT26 tumour cells have very low levels of endogenous CEBPA mRNA) and an increased expression of CD8a and Granzyme A mRNA are consistent with an increased level of activated CD8 T-cells in the combination group compared to either PD-1 antibody or MTL-CEBPA treatment alone.

### Example 8

### MLT-CEBPA and Combination Treatment in Sorafenib-Resistant Tumour Mice

This study was aimed to evaluate the in vivo efficacy of MTL-CEBPA in a combination treatment with anti-PD1 or Nexavar® (Sorafenib) in a hepatocellular carcinoma adenocarcinoma (BNL) subcutaneous xenograft model in BALB/c nude mice.

### Materials and Methods

Cell Culture: The mouse hepatocellular carcinoma cell line BNL was maintained in Dulbecco's modified Eagle's medium (DMEM, Gibco by Invitrogen) with 250 ng /mL G418 (Merck, Germany), 1% antibiotic antimycotic (Gibco by Invitrogen) and 10% fetal bovine serum (Gibco by Invitrogen) and cultured at 37° C. in a humidified atmosphere.

Tumour Inoculation and Experimental design: Each BALB/c mice were injected subcutaneously (s.c.) on the flank with 3×10⁵ BNL-Luc cells in 0.05 ml of PBS. After inoculation for 3 weeks, Nexavar® (Sorafenib) was orally administered every day for two weeks (30 mg/kg/day).

The ‘refractory animals’ where visible tumour nodules showed a 20% increase in measurable dimension when compared to control were selected. The selected animals were randomized into the 7 treatment groups (Table 18).

Compounds:
PBS: UniRegion Bio-Tech. Cat: UR-PBS001-5L
MTLCEBPA: MINA tx: MIT0615A
Nexavar® (Sorafenib): Bayer HealthCare Pharmaceuticals
Anti-PD-1: InVivoMAb anti-mouse PD-1: BioXCell. Cat No: BE0146 /717918O1

### Groups and Treatments for Combination Study

60 mice were injected with 3×10⁵ of BNL-Luc cells in 0.5 mL mixture of PBS. After 3 weeks, the mice were dosed with 30 mg/kg of Sorafenib p.o. every day for 2 weeks. 42 mice that showed a 20% in-crease in tumour volume were selected and assigned into 7 groups using randomized block design with 6 mice in each group. The 7 groups included PBS as Control group [PBS]; MTL-CEBPA [C], (4.2 mg/ml, I.V., d1, d3, d5); anti-PD1 antibody [P] (250 ug, I.P., d1, d3, d5); Nexavar® (Sorafenib) [N] (30 mg/kg, P.O. daily); MTL-CEBPA+anti-PD1 [C+P]; MTL-CEBPA+Nexavar® (Sorafenib) [C+N] and MTL-CEBPA+anti-PD1+Nexavar® (Sorafenib) [C+P+N].

TABLE 18
Treatment groups

| | | | | | Dosing | |
|---|---|---|---|---|---|---|
| Group | n | Treatment | Dose | | Route | Schedule |
| 1 = PBS | 6 | Control | — | | IV | (d1, d3, d5) |
| | | | | | | for 3 weeks |
| 2 = C | 6 | MTL- | 4.2 | mg/kg | IV | (d1, d3, d5) |
| | | CEBPA | | | | for 3 weeks |
| 3 = P | 6 | anti-PD-1 | 250 | ug | IP | (d1, d3, d5) |
| | | antibody | | | | for 3 weeks |
| 4 = N | 6 | Sorafenib | 30 | mg/kg | PO | Daily for 3 weeks |
| 5 = C + P | 6 | MTL- | 4.2 | mg/kg | IV | (d1, d3, d5) |
| | | CEBPA | | | | for 3 weeks |
| | | Anti-PD1- | 250 | ug | IP | (d1, d3, d5) |
| | | antibody | | | | for 3 weeks |
| 6 = C + N | 6 | MTL- | 4.2 | mg/kg | IV | (d1, d3, d5) |
| | | CEBPA | | | | for 3 weeks |
| | | Sorafenib | 30 | mg/kg | PO | Daily for 3 weeks |
| 7 = C + | 6 | MTL- | 4.2 | mg/kg | IV | (d1, d3, d5) |
| P + N | | CEBPA | | | | for 3 weeks |
| | | Anti-PD1- | 250 | ug | IP | (d1, d3, d5) |
| | | antibody | | | | for 3 weeks |
| | | Sorafenib | 30 | mg/kg | PO | Daily for 3 weeks |

Note:
Dosing volume: adjusted dosing volume based on body weight 10 μL/g.
Grouping day was day 1 (d1), and treatment was started on day 1 (d1).

### Results and Discussion

First, whether single agent treatments with the tyrosine kinase inhibitor, Nexavar (Sorafenib), the immune checkpoint blockade (anti-PD-1 antibody) or MTL-CEBPA alone would result in tumour shrinkage in the Nexavar resistant BNL xenograft mice was investigated.

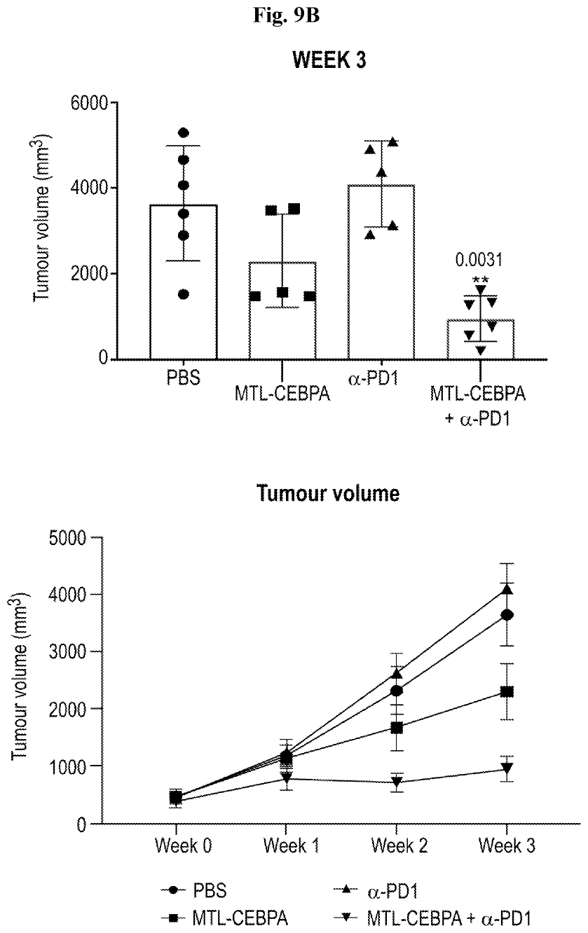

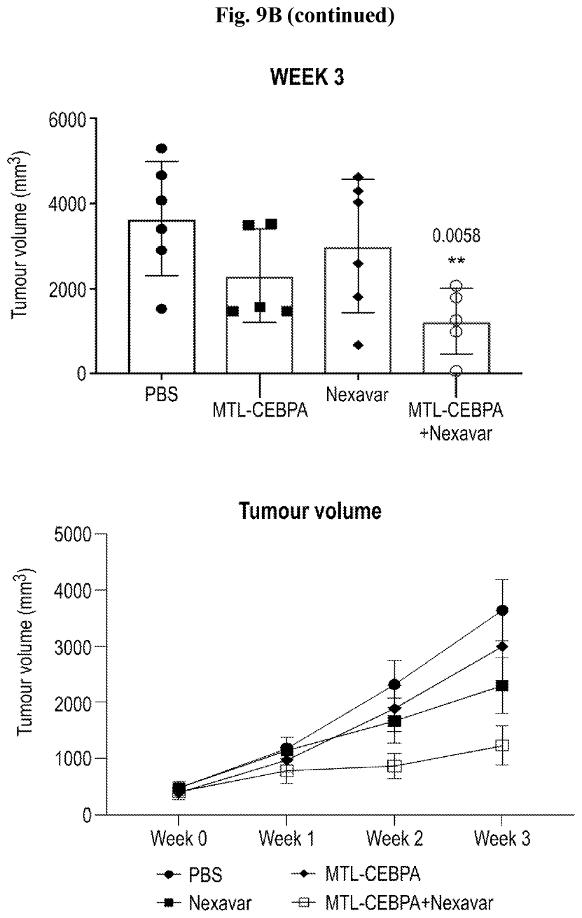

Mice treated with MTL-CEBPA alone showed a significant 49% reduction in tumour weight (p=0.01) (FIG. 9A) and a 37% reduction in tumour volume (FIG. 9B) after 3 weeks of treatment. Anti-PD1 treatment alone or Nexavar treatment alone showed no effect on tumour volume or weight (FIG. 9A and FIG. 9B).

In the animal groups that received combination treatment of either Nexavar or anti-PD1 with MTL-CEBPA, a drastic and significant reduction in tumour volume and weight was observed. MTL-CEBPA combined with anti-PD1 showed a 74% decrease in tumour volume (p<0.004) (FIG. 9B) and 83% reduction in tumour weight (p<0.002) (FIG. 9A). MTL-CEBPA combined with Nexavar showed 66% reduction in tumour volume (p<0.006) (FIG. 9B) and 80% reduction in tumour weight (p=0.001) (FIG. 9A). Animals treated with MTL-CEBPA+Nexavar+anti-PD1 showed a 77% re-duction in tumour volume and 87% reduction in tumour weight and this activity was greater than either of the double combinations.

To asses if there was synergistic effect of the tested compounds, MTL-CEBPA was combined with anti-PD1 and Nexavar in parallel. After 3 weeks of treatment the anti-tumour response of combining MTL-CEBPA with anti-PD1 showed a 65% reduction in tumour growth rate. MLT-CEBPA combined with Nexavar showed a 62% reduction in tumour growth rate. When all three compounds were administered at the same time, there was a 76% reduction in tumour growth rate (p=0.003), relative to untreated animals;

### Conclusions

It has been demonstrated with strong evidence that upregulation of CEBPA by MTL-CEBPA when combined with standard of care anti tumour compounds Nexavar and/or anti-PD1 promotes a strong and consistent anti-tumour response. saRNA induced upregulation of the tumour suppressor CEBPA gene significantly improves the anti-tumour capacity of standard chemotherapy agents.

### Equivalents and Scope

Those skilled in the art will recognize or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A pharmaceutical composition comprising a synthetic isolated saRNA and at least one additional active agent, wherein the saRNA up-regulates the expression of the C/EBPα gene, wherein the saRNA comprises a strand that is at least 80% complement to a region on SEQ ID No. 3, and wherein the strand has 14-30 nucleotides.

2. The pharmaceutical composition of claim 1, wherein the saRNA is double-stranded and comprises an antisense strand and a sense strand.

3. The pharmaceutical composition of claim 2, wherein the antisense strand of the saRNA comprises a sequence of SEQ ID No. 1 (CEBPA-51).

4. The pharmaceutical composition of claim 3, wherein the sense strand of the saRNA comprises a sequence of SEQ ID No. 2 (CEBPA-51).

5. The pharmaceutical composition of claim 1, wherein the additional active agent impacts FGFR4 signaling.

6. The pharmaceutical composition of claim 5, wherein the additional active agent is an FGFR4 inhibitor.

7. The pharmaceutical composition of claim 6, wherein the additional active agent is a small inhibiting RNA (FGFR4-siRNA), an FGFR4 antagonist antibody, or a small molecule FGFR4 inhibitor.

8. The pharmaceutical composition of claim 1, wherein the additional active agent reduces CEBPB expression.

9. (canceled)

10. The pharmaceutical composition of claim 1, wherein the additional active agent is a checkpoint inhibitor or an immune checkpoint blockade agent.

11. The pharmaceutical composition of claim 10, wherein the additional active agent is an inhibitor of CTLA4, PD-1 or PD-L1.

12. The pharmaceutical composition of claim 11, wherein the active agent is a PD-1 antibody.

13. The pharmaceutical composition of claim 1, wherein the additional active agent is a tyrosine kinase inhibitor.

14. The pharmaceutical composition of claim 13, wherein the tyrosine kinase inhibitor is sorafenib or lenvatinib or a combination thereof.

15. (canceled)

16. The pharmaceutical composition of claim 1, wherein the composition further comprises a tyrosine kinase inhibitor and a checkpoint inhibitor.

17. The pharmaceutical composition of claim 16, wherein the tyrosine kinase inhibitor is sorafenib and the checkpoint inhibitor is a PD-1 inhibitor.

18. A method of up-regulating the expression of the C/EBPα gene in a cell, comprising administering a synthetic isolated saRNA and at least one additional active agent, wherein the saRNA up-regulates the expression of the C/EBPα gene, wherein the saRNA comprises a strand that is at least 80% complement to a region on SEQ ID No. 3, and wherein the strand has 14-30 nucleotides.

19. The method of claim 18, wherein the saRNA is double-stranded and comprises an antisense strand and a sense strand.

20. The method of claim 19, wherein the antisense strand of the saRNA comprises a sequence of SEQ ID No. 1 (CEBPA-51).

21. The method of claim 20, wherein the sense strand of the saRNA comprises a sequence of SEQ ID No. 2 (CEBPA-51).

22. The method of claim 18, wherein the additional active agent reduces FGFR4 levels.

23. The method of claim 22, wherein the additional active agent is an FGFR4 inhibitor.

24. The method of claim 23, wherein the additional active agent is a small inhibiting RNA (FGFR4-siRNA), an FGFR4 antagonist antibody, or a small molecule FGFR4 inhibitor.

25. The method of claim 18, wherein the saRNA is administered simultaneously or sequentially with the additional active agent.

26. (canceled)

27. (canceled)

28. (canceled)

29. A method of treating cancer, liver fibrosis, liver failure, or nonalcoholic steatohepatitis (NASH) of a subject in need thereof, comprising administering a synthetic isolated saRNA and at least one additional active agent, wherein the saRNA up-regulates the expression of C/EBPα gene, wherein the saRNA comprises a strand that is at least 80% complement to a region on SEQ ID No. 3, and wherein the strand has 14-30 nucleotides.

30. The method of claim 29, wherein the saRNA is double-stranded and comprises an antisense strand and a sense strand.

31. The method of claim 30, wherein the antisense strand of the saRNA comprises a sequence of SEQ ID No. 1 (CEBPA-51).

32. The method of claim 30, wherein the sense strand of the saRNA comprises a sequence of SEQ ID No. 2 (CEBPA-51).

33. The method of claim 29, wherein the saRNA is administered as MTL-CEBPA.

34. The method of claim 29, wherein the saRNA is administered simultaneously or sequentially with the additional active agent.

35. The method of claim 29, wherein the additional active agent reduces FGFR4 levels.

36. The method of claim 35, wherein the additional active agent is an FGFR4 inhibitor.

37. The method of claim 36, wherein the additional active agent is a small inhibiting RNA (FGFR4-siRNA), an FGFR4 antagonist antibody, or a small molecule FGFR4 inhibitor.

38. (canceled)

39. (canceled)

40. The method of claim 29, wherein the additional active agent is a checkpoint inhibitor or an immune checkpoint blockade agent.

41. The method of claim 40, wherein the additional active agent is an inhibitor of CTLA4, PD-1 or PD-L1.

42. The method of claim 41, wherein the additional active agent is a PD-1 antibody.

43. The method of claim 29, wherein the additional active agent is a tyrosine kinase inhibitor.

44. The method of claim 43, wherein the tyrosine kinase inhibitor is sorafenib or lenvatinib or a combination thereof.

45. (canceled)

46. The method of claim 45, wherein sorafenib is administered concomitant or post saRNA treatment.

47. The method of claim 29, wherein the subject further receives Radiofrequency ablation (RFA) treatment.

48. The method of claim 47, wherein the subject receives RFA treatment prior to saRNA treatment.

49. The method of claim 29, wherein the subject further receives a tyrosine kinase inhibitor treatment and a checkpoint inhibitor treatment.

50. The method of claim 49, wherein the tyrosine kinase inhibitor is sorafenib and the checkpoint inhibitor is a PD-1 inhibitor.

51. The method of claim 29, wherein the subject has cancer.

52. The method of claim 51, wherein the cancer is selected from hepatocellular carcinoma (HCC), colorectal cancer, gastric cancer, skin cancer, pancreatic cancer, head and neck cancer, cervical cancer, and prostate cancer.


SEQUENCE LISTING (7 sequences)

SEQ ID NO: 1 - RNA, 21 nt, Artificial Sequence
  Description of Artificial Sequence Synthetic oligonucleotide
  modified_base (20)..(21): 2′-OMe modified nucleotide
gaccagugacaaugaccgcuu

SEQ ID NO: 2 - RNA, 21 nt, Artificial Sequence
  Description of Artificial Sequence Synthetic oligonucleotide
  modified_base (1)..(1): Inverted abasic modification
  modified_base (1)..(2): 2′-OMe modified nucleotide
  modified_base (4)..(4): 2′-OMe modified nucleotide
  modified_base (10)..(10): 2′-OMe modified nucleotide
  modified_base (14)..(14): 2′-OMe modified nucleotide
  modified_base (20)..(21): 2′-OMe modified nucleotide
gcggucauugucacuggucuu

SEQ ID NO: 3 - DNA, 4001 bp, Homo sapiens
tccctctcccaccaggggtatacatcctcagagctgacccacgacctagctttctggtgtgactcggggtgggggctcccactggtcacctggtgacccccatcgcagtgagttccgccccaaggggaagcccagcctatagcaggctggggtggggtgtgtgcggagggaggtgggagaggcgtggaactagagaccctccaccttcatgtagaactaggggaacaaccttaggttccaagccccaagtccctatgtttccacccctttctaaggacaggcgtggaggagcggctggggctggcgggcttgtcgggatctcagctccctgagccctcctcctgccacgggcctgctcccctccttctctcatgggggtctgctgtagcctcgggaaggaggcaggaaacctccaaataaaatgacaaggcacgatttgctccccctactcagtaggcattggagcggtgagtttgcatttccaaggcacaaggttatcctaaatactagagttgccgggctcccagctcagccccaagaattctcccctcctcgcagggagaagccaccgcctggccccctcatcttagacgcaccaagtccggcgcagaggaagggaggggacacgcggagcaggccaggctttcaggaggcaccggaatctcctagtcctggctcgcacggctcgggcaagcctcgagatccggcgaccccaaaccactccctgggtccccgccggaggctggcccagggcggtcccacagccgcgcgcctcacgcgcagttgcccatggccttgaccaaggagctctctggcagctggcggaagatgccccgcagcgtgtccagttcgcggctcagctgttccacccgcttgcgcaggcggtcattgtcactggtcagctccagcaccttctgctgcgtctccacgttgcgctgcttggccttgtcgcggctcttgcgcaccgcgatgttgttgcgctcgcgccgcacccggtactcgttgctgttcttgtccaccgacttcttggccttgcccgcgccgctgccgccactcgcgcggaggtcggggtgcgcggcgcccagccccttgagcgcgctgccagggcccggcaggccggcggcaccgagcgcgggcgcggggtgcgggctgggcacgggcgtgggcggcggcgtggggtgaccgggctgcaggtgcatggtggtctggccgcagtgcgcgatctggaactgcaggtgcggggcggccaggtgcgcgggcggcgggtgcgggtgcgggtgcgagggcggcggcggcggcggcggctggtaagggaagaggccggccagcgccagctgcttggcttcatcctcctcgcggggctcctgcttgatcaccagcggccgcagcgccggcgccccgacgcgctcgtacaggggctccagcctgccgtccaggtagccggcggccgcgcagccgtagccgggcgggggcccgtgcgctcccccgggcatgacggcgccgccggggcccgcgggcgcgcccgggtagtcaaagtcgccgccgccgccgccgcccgtggggcccacggccgccttggccttctcctgctgccggctgtgctggaacaggtcggccaggaactcgtcgttgaaggcggccgggtcgatgtaggcgctgatgtcgatggacgtctcgtgctcgcagatgccgcccagcggctccggggcggcaggtggggcgggaggctgcgcggggcccgcgccccggggaaagccgaaggcggcgctgctgggcgcgtgcggggggctctgcaggtggctgctcatcgggggccgcggctccgcctcgtagaagtcggccgactccatgggggagttagagttctcccggcatggcgagcctcggcggcctccagcctgcgcggggcgtcgccgccgcccacccggagaccctgctcgcccgcgcccgcgcacctccgggtcgcgaatggcccggcccgcgccggcccagcttttatacccggcaggccgcgtcgccccctagagtccgaggcggcctctgtccccgggctgcggcggcgcggcgcctgctgggtcctagcgcgcggccggcatggggcggcgaaccagcgcggcacagcgccgcgctccccaggcaggccgcggcgcaacgcccaccgcctccagcgcgcccagcagagccgcggcgctcgctccaagctccgcccccggcccggccgtcgcccccgcgcccacgtggtcggtagcgggggccccctcctcctgcctgccctaggcgcccgtatccagccacggccgggagcccaggagtatcccgaggctgcacggggtaggggtggggggcggagggcgagtcttggtcttgagctgctggggcgcggattctctttcaaagccagaaccaggcctgtcccggacccgcgtcccggggaggctgcagcgcagagcagcggggctggggccggtggggggccgtttgggacgcgcggagaggtcctgagcgcggtggctctgcgtctcctagctctgatctccaggctacccctgtgattccgcgcagaggtacctctcggaggacgccggggtcccatgggcggcgccgcgcagggcgctaggaccccgcggggagcggaggcggcctcggcccgggagcctggaggacctggccggtcgatccgcccgggctggaaaactttctttataattacttctccaggtcggagcgcgcggcttgctaggcgcgcggggccggcgctgttacccggcgtggagtcgccgattttttttcctgcgggaccgcggggccccccagactagcggagctggacgccggggcgagcacggggaggggcgcaccgagggaggagacaaacttaactctggggccgggattccgaggcgggggccgcagccctcgaggcccgaagccaccgcttcctcccccgcctccccattcaggtgggcgccaacggcgggagcgagggtgtccaggccgccgggctgccaggtccgagcacgcacagggagaactctgcccagtggttcgccgggcgctgtagtccccgggatcctagggaccgaggcggccaggccctggggccccttgagtgcggcagctaatgctctcaccgcggcgggggaaggagcttgccaccgagacccccagccacgtgcgtccctcgcattctttaccggggccggggtggcggctacggaccgtcagctgggcccagatggagtcttgggagccctcaagtgtctcctgtccttgcccgcgccgcccctcgccactggcgctgaggcctgacgccgcctgcgtcccggctagaggcgcgcttgcctacaggtgagggaagacccccttcaccgacagtggccttaggcctggcaaggcgccacgacccgcccaggagccccggagggggcacagctaaaaacaccgctggagagccccgagcttccacgacgatcgcagtaaagaagcagtttcatctgggcaacgcacactgcgctttaatcaagttcctattcaacatagtcccagtgattaatagcccaactgcttcgttttcggtccagagctcataaacaagatatttttagcttgacgcttttggacgggagggagtaaaaaccagatacgttaaataaatatcccgatgtgagccggagagctgcttgctgagccaaatgcaggacccattcatatagcattcacctgtggagggagacctggacggaaatcaaaaagcaccaagagcgatttgcgtttttttctgcggtgctaaaactaatggcttttcctacctaggaacaaagaaacgccactgtacatgcacggttcccggcctgtggagttgtgggaggaaggcgatgtctggccttttttgcacagctgctgttgcctgcccagagatcgggaactctgccccgtaggactggaagaaacctcagtaatgggaataagactttgtccaatagggggctgatgaatgtgtg

SEQ ID NO: 4 - DNA, 21 bp, Artificial Sequence
  Description of Artificial Sequence Synthetic oligonucleotide
  Description of Combined DNA/RNA Molecule Synthetic oligonucleotide
cauugacuacuauaagaaatt

SEQ ID NO: 5 - DNA, 21 bp, Artificial Sequence
  Description of Artificial Sequence Synthetic oligonucleotide
  Description of Combined DNA/RNA Molecule Synthetic oligonucleotide
uuucuuauaguagucaaugtg

SEQ ID NO: 6 - DNA, 21 bp, Artificial Sequence
  Description of Artificial Sequence Synthetic oligonucleotide
  Description of Combined DNA/RNA Molecule Synthetic oligonucleotide
  modified_base (2)..(3): 2′-OMe modified nucleotide
  modified_base (5)..(5): 2′-OMe modified nucleotide
  modified_base (7)..(8): 2′-OMe modified nucleotide
  modified_base (12)..(12): 2′-OMe modified nucleotide
  modified_base (14)..(17): 2′-OMe modified nucleotide
  misc_feature (20)..(21): Phosphorothioate linkage
cuuacgcugaguacuucgatt

SEQ ID NO: 7 - DNA, 21 bp, Artificial Sequence
  Description of Artificial Sequence Synthetic oligonucleotide
  Description of Combined DNA/RNA Molecule Synthetic oligonucleotide
  modified_base (7)..(7): 2′-OMe modified nucleotide
  modified_base (11)..(11): 2′-OMe modified nucleotide
  modified_base (16)..(16): 2′-OMe modified nucleotide
  misc_feature (20)..(21): Phosphorothioate linkage
ucgaaguacucagcguaagtt